(19) United States
(12) Patent Application Publication
(10) Pub. No.: US 2005/0209629 A1
(43) Pub. Date: Sep. 22, 2005

(54) Resorbable containment device and process for making and using same

(76) Inventors: Sean H. Kerr, Oreland, PA (US); Stuart Weikel, Drexel Hill, PA (US); James W. Dwyer, West Chester, PA (US)

Correspondence Address: JONES DAY, 222 EAST 41ST STREET, NEW YORK, NY, 10017-6702, US

(21) Appl. No.: 11/067,438

(22) Filed: Feb. 25, 2005

Related U.S. Application Data
(63) Continuation-in-part of application No. 10/636,549, filed on Aug. 8, 2003.
Application No. 10/636,549 is a continuation of application No. 09/908,899, filed on Jul. 20, 2001, now Pat. No. 6,632,235.
(60) Provisional application No. 60/284,510, filed on Apr. 19, 2001.

(51) Int. Cl. A61M 29/00

(52) U.S. Cl. 606/192

(57) ABSTRACT

The present invention is directed to a resorbable containment device for use in treating voids in bone and restoring the anatomy of diseased or fractured bone. The resorbable containment device may be a balloon of varied size or shape to conform to the bone to be treated and may be deployed in any type of bone where collapsed fractures of cortical bone may be treated by restoring the bone from its inner surface. The containment device may be formed from a pluronic based polymer and may degrade in-vivo within a period of weeks after implantation into bone. The balloon may have multiple layers to provide desired surface characteristics, resistance to puncture and tearing, or other beneficial properties, as appropriate for the particular application of the device.

### CROSS-REFERENCE TO RELATED APPLICATIONS

This is a continuation-in-part of pending U.S. patent application Ser. No. 10/636,549, filed Aug. 8, 2003, which further claims the benefit under 35 U.S.C. § 120 of U.S. patent application Ser. No. 09/908,899, filed Jul. 20, 2001, now U.S. Pat. No. 6,632,235 B2, which claims the benefit under 35 U.S.C. § 119(e) of Provisional Application No. 60/284,510, filed Apr. 19, 2001. All the foregoing documents are herein incorporated by reference.

### FIELD OF THE INVENTION

The present invention is directed to a containment device for filling voids in bone, a process for making a containment device for filling voids in bone, and a method for use in orthopedic procedures to treat bone, and in particular to an improved device and method for reducing fractures in bone and treatment of the spine.

### BACKGROUND OF THE INVENTION

Medical balloons are commonly known for dilating and unblocking arteries that feed the heart (percutaneous translumenal coronary angioplasty) and for arteries other than the coronary arteries (noncoronary percutaneous translumenal angioplasty). In angioplasty, the balloon is tightly wrapped around a catheter shaft to minimize its profile, and is inserted through the skin and into the narrowed section of the artery. The balloon is inflated, typically, by saline or a radiopaque solution, which is forced into the balloon through a syringe. Conversely, for retraction, a vacuum is pulled through the balloon to collapse it.

Medical balloons also have been used for the treatment of bone fractures. One such device is disclosed in U.S. Pat. No. 5,423,850 to Berger, which teaches a method and an assembly for setting a fractured tubular bone using a balloon catheter. The balloon is inserted far away from the fracture site through an incision in the bone, and guide wires are used to transport the uninflated balloon through the medullary canal and past the fracture site for deployment. The inflated balloon is held securely in place by the positive pressure applied to the intramedullary walls of the bone. Once the balloon is deployed, the attached catheter tube is tensioned with a calibrated force measuring device. The tightening of the catheter with the fixed balloon in place aligns the fracture and compresses the proximal and distal portions of the fractured bone together. The tensioned catheter is then secured to the bone at the insertion site with a screw or similar fixating device.

As one skilled in the related art would readily appreciate, there is a continuing need for new and innovative medical balloons and balloon catheters, and in particular a need for balloon catheter equipment directed toward the treatment of diseased and damaged bones. More specifically, there exists a need for a low profile, high-pressure, puncture and tear resistant medical balloon, that can be used to restore the natural anatomy of damaged cortical bone.

### SUMMARY OF THE INVENTION

The present invention relates to a device for containing material inside bone. The device may include a barrier member configured and adapted for insertion into bone, the barrier member having inner and outer surfaces, the inner surface defining a space. The barrier member may be capable of preventing fluid within the space from passing through the inner surface to the outer surface. The barrier member may comprise a polyurethane polymer based on capralactone, such that barrier member is capable of degrading into biologically compatible substances in vivo. The barrier member may include a plurality of polymer layers. Additionally, the barrier member may comprise a polyurethane polymer based on caprolactone and pluronic. The barrier member may have a tensile strength between about 15 and about 50 MPa. For example, the barrier member may have a tensile strength between about 25 and about 35 MPa. The barrier member may have a Young's modulus of between about 5 and 30. In an exemplary embodiment, the barrier member may have a Young's modulus of between about 15 and 25. The barrier member may have an elongation at break of between about 600 and 1000. For instance, the barrier member may have an elongation at break of between about 850 and 950. The barrier member may have an average molecular weight of between about 100,000 and 200,000 dalton. For instance, the barrier member may have an average molecular weight of between about 150,000 and 190,000 dalton. The mass may degrade in vivo after the device is implanted into bone or another part of the body. In an illustrative embodiment, more than about 60-percent of the mass degrades after about 16 weeks of in vivo degradation. The mass may degrade in vivo to produce carbon dioxide, water and diamine. In a non-limiting example, the barrier member may be about 0.3 mm in thickness.

The present invention is also directed to methods for treating voids in bone. One method may include accessing a cavity in bone, where the cavity has one or more boundary surfaces and may contain organic material. The cavity then may be prepared to be substantially clear from organic material. Boundary surfaces of the cavity may be spray coated with a sealant prior to filing of the cavity with a filler material. The method may further include irrigating the cavity and boundary surfaces to remove organic material and cancellous bone. Irrigating the cavity may also wash the boundary surfaces. The cavity may be aspirated to clear liquid and solid materials from the cavity and the boundary surfaces. The method may include deploying a solid barrier formed of biologically resorbable material on the boundary surfaces and placing a liquid bone filler against the barrier to fill the cavity. The method may also include preventing the transport of foreign materials from the cavity. This may involve occluding openings in the boundary surfaces and occluding voids in cancellous bone. The method may further include occluding vascular passageways in boundary surfaces of the cavity. The method may further include occluding cracks in cortical bone, where the cracks may extend into cortical bone. The method may further use an instrument to place sealant on the boundary surfaces of the cavity. The method also may include containing liquid bone filler material in the solid barrier member to form a containment device. The containment device may be filled with bone filler material. The containment device may be filled with bone filler material to substantially fill the cavity. The method may further comprise allowing the bone filler material to cure. The containment device may be implanted within the bone. Spatial relationships between the containment device and the cavity may be interpreted by fluroscopic imagery. One or more containment devices may be implanted within the bone. Enhancing the quality of fluroscopic imagery may be accomplished by assigning a distinctive radiographic signature to each containment device implanted within the bone.

### BRIEF DESCRIPTION OF THE DRAWINGS

Preferred features of the present invention are disclosed in the accompanying drawings, wherein similar reference characters denote similar elements throughout the several views, and wherein:

FIG. 1 shows a perspective view of a medical balloon catheter system according to the present invention.

FIG. 2 shows a perspective view of the balloon of FIG. 1 FIG. 3 shows a side view of a balloon of the present invention.

FIG. 4 shows a partial sectional view along the longitudinal axis of another embodiment of the balloon catheter of FIG. 1.

FIG. 5 shows a partial sectional view along the longitudinal axis of another embodiment of the balloon catheter of FIG. 1.

FIG. 6 shows a partial sectional view along the longitudinal axis of another embodiment of the balloon catheter of FIG. 1.

FIG. 7 shows a partial sectional view along the longitudinal axis of another embodiment of the balloon catheter of FIG. 1.

FIG. 8 shows a partial sectional view along the longitudinal axis of another embodiment of the balloon catheter of FIG. 1.

FIG. 9 shows a partial sectional view along the longitudinal axis of another embodiment of the balloon catheter of FIG. 1.

FIG. 10 shows a sectional view perpendicular to the longitudinal axis of the balloon of FIG. 9.

FIG. 11 shows a partial sectional view along the longitudinal axis of another embodiment of the balloon catheter of FIG. 1.

FIG. 12 shows a sectional view perpendicular to the longitudinal axis of the balloon of FIG. 11.

FIG. 13 shows a perspective view of another embodiment of the balloon of FIG. 1.

FIG. 14 shows a perspective view of another embodiment of the balloon of FIG. 1.

FIG. 15 shows an elevation view of another embodiment of the balloon of FIG. 1.

FIG. 16 shows an elevation view of another embodiment of the balloon of FIG. 1.

FIG. 17 shows a plan view of the catheter of FIG. 13.

FIG. 18 shows a plan view of an reinforcing insert for the catheter of FIG. 17.

FIG. 19 shows an enlarged sectional view along line 19—19 of FIG. 18.

FIG. 20 shows a enlarged sectional view along line 19—19 of another embodiment of the reinforcing insert of FIG. 18.

FIG. 21 shows an enlarged sectional view along line 19—19 of another embodiment of the reinforcing insert of FIG. 18.

FIG. 22 shows an enlarged sectional view along line 19—19 of another embodiment of the reinforcing insert of FIG. 18.

FIG. 23 shows an enlarged sectional view along line 19—19 of another embodiment of the reinforcing insert of FIG. 18.

FIG. 24 shows an enlarged sectional view along line 19—19 of another embodiment of the reinforcing insert of FIG. 18.

FIG. 25 shows a perspective view of an exemplary embodiment of a reinforcing insert for the catheter of FIG. 17.

FIG. 26 shows a partial sectional view along the longitudinal axis of another embodiment of the balloon catheter of FIG. 1.

FIG. 27 shows a sectional view through line 27-27 of FIG. 26

FIG. 28 shows a perspective view of another embodiment of the balloon of FIG. 1.

FIG. 29 shows a perspective view of another embodiment of the balloon of FIG. 1.

FIG. 30 shows an intermediate perspective view of another embodiment of the balloon of FIG. 1.

FIG. 31 shows a partial sectional view along the longitudinal axis of the fully constructed balloon of FIG. 30.

FIG. 32 shows a schematic representation of the catheter construction of the balloon of FIG. 30.

FIG. 33 shows a perspective view of another embodiment of the balloon of FIG. 1.

FIG. 34 shows a perspective view of another embodiment of the balloon of FIG. 1.

FIG. 35 shows a perspective view of another embodiment of the balloon of FIG. 1.

FIG. 36 shows a plan view of another embodiment of the balloon catheter of FIG. 1.

FIG. 37 shows a sectional view through line 37-37 of FIG. 36.

FIG. 38 shows a perspective view of an exemplary embodiment of a resorbable containment device.

FIG. 39 shows a perspective view of a mandrel for forming the resorbable containment device of FIG. 38.

FIG. 40 shows a perspective view of an embodiment of the resorbable containment device of FIG. 38 with a strand.

FIG. 41 shows a perspective view of another embodiment of the resorbable containment device of FIG. 38 with a strand.

FIG. 42 shows a perspective view of an exemplary embodiment of a strand for use with the mandrel of FIG. 39 in forming a resorbable containment device.

FIG. 43 shows a perspective view of another embodiment of a strand for use with the mandrel of FIG. 39 in forming a resorbable containment device.

### DETAILED DESCRIPTION OF THE PREFERRED EMBODIMENTS

In the description that follows, any reference to either orientation or direction is intended primarily for the convenience of description and is not intended in any way to limit the scope of the present invention thereto.

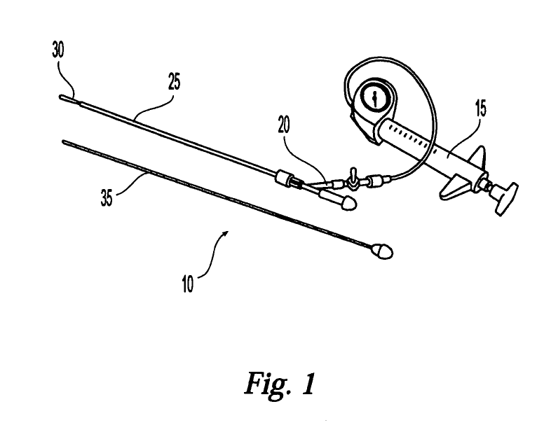

FIG. 1 shows an apparatus 10 for use in reducing bone fractures according to the method of the present invention. The apparatus 10 comprises an inflation device 15, y-connector 20, catheter shaft 25, balloon 30, and hub shaft 35. As shown in FIG. 1, The balloon 10 is shown at the distal end of the catheter shaft 25, prior to deployment. The instruments illustrated in FIG. 1 are representative of the tools and other devices that may be used in conjunction with the balloon. These tools, however, may not always be required or may be replaced by different devices that perform similar, additional, or different functions. For example, one of ordinary skill in the art would appreciate that the y-connector 20 may be replaced by a wide variety of other suitable devices.

The balloon 30 may be used to treat any bone with an interior cavity sufficiently large enough to receive the balloon 30. Non-limiting examples of bones that are suitable candidates for anatomical restoration using the device and method of the present invention include vertebral bodies, the medullary canals of long bones, the calcaneus and the tibial plateau. The balloon 30 can be designed and adapted to accommodate particular bone anatomies and different cavity shapes, which may be made in these and other suitably large bones.

Additionally, the balloon may be designed and configured to be deployed and remain in the bone cavity for an extended period of time. For instance, the balloon may be inflated with natural or synthetic bone filler material or other suitable inflation fluid once the balloon is located within the bone cavity. Once filled, the balloon is allowed to remain within the bone for a prescribed period or perhaps indefinitely. The duration of time that the balloon remains within the bone may depend upon specific conditions in the treated bone or the particular objective sought by the treatment. For example, the balloon may remain within the cavity for less than a day, for several days, weeks, months or years, or even may remain within the bone permanently. As explained in greater detail below, the balloon may also be adapted to serve as a prosthetic device outside of a specific bone cavity, such as between two adjacent vertebrae.

In addition, the outer surface of the balloon may be treated with a coating or texture to help the balloon become more integral with the surrounding bone matter or to facilitate acceptance the balloon by the patient. The selection of balloon materials, coatings and textures also may help prevent rejection of the balloon by the body. The inner surface of the balloon likewise may be textured or coated to improve the performance of the balloon. For instance, the inner surface of the balloon may be textured to increase adhesion between the balloon wall and the material inside.

In yet another embodiment, the balloon may be designed to rupture, tear or otherwise open after the filler material injected inside the balloon has set up or sufficiently gelled, cured or solidified. The balloon may then be removed from the bone while leaving the filler material inside. This approach may result in a more controlled deployment of bone filler material to a treated area. It also may allow the bone filler material to be at least partially preformed before being released into the bone. This may be particularly beneficial where leakage of bone filler material out of damaged cortical bone may be a concern, although there may be other situations where this configuration would also be beneficial.

Alternatively, the balloon may be opened or ruptured in a manner that would permit the filler material to allow the inflation fluid to be released into the cavity. For instance, the opening of the balloon may be predetermined so that the flow of filler material travels in a desired direction. Moreover, the filler material may be held within the balloon until it partially sets so that, upon rupture of the balloon, the higher viscosity of the filler material limits the extent to which the filler material travels.

The balloon also may be designed and configured to release inflation fluid into the cavity in a more controlled fashion. For instance, the balloon catheter might be provided with a mechanism to initiate the rupture process in a highly controlled fashion. In one embodiment, predetermined seams in the balloon might fail immediately and rupture at a certain pressure. In another embodiment, the seams might fail only after prolonged exposure to a certain pressure, temperature, or material.

One skilled in the art would appreciate any number of ways to make the balloon open or rupture without departing from the spirit and scope of the present invention. For example, at least a portion of the balloon may be dissolved until the filler material is released into the bone cavity. In another example, the balloon may rupture and become harmlessly incorporated into the inflation fluid medium. In yet another example, the filler material may be designed to congeal when contacted to a chemical treatment applied to the surface of the balloon. In yet another embodiment, two balloons (or a single balloon having two chambers) may be designed and configured to release a combination of fluids that when mixed together react to form an inert filler material within the cavity. In another embodiment, different areas of the seam or balloon might be designed to rupture at different predetermined pressures or at different times.

Further, the balloon may be designed to be opened in any number of ways. For instance, a surgeon may lyse the balloon once the desired conditions of the bone filler material are reached. A balloon adapted to rupture and release inflation fluid into direct contact with a cavity also may be designed and configured to split along predetermined seams. The seams might run parallel to the longitudinal axis of the balloon and remain secured to the catheter at the proximal tip of the balloon, resembling a banana peel which has been opened. In another embodiment, the predetermined seams might consist of a single spiraling seam originating form the distal tip of the balloon and ending at the proximal tip of the balloon, resembling an orange peel which has been opened.

Other balloon adaptations may be provided to lyse the balloon in a controlled fashion. For instance, a balloon may be constructed with failure zones that are adapted so that structural failure under a triggering condition would occur preferentially in a localized area. For instance, a balloon might have a failure zone comprising a thinner membrane. In another example, the balloon might be designed to lack tensile reinforcing elements in a particular region. In yet another example, a region of the balloon might be comprised of a material that would fail due to a chemical reaction. For instance, the chemical reaction may be an oxidation or reduction reaction wherein the material might sacrificially neutralize a weak acid or base. In another example, the sacrificial region might comprise a pattern of pore like regions. This sacrificial region may comprise a specific pattern of pores that might form a latent perforation in the balloon membrane or may be randomly distributed in a localized area.

The ruptured balloon may then be removed from the bone cavity, leaving behind the deployed bone filler material. To facilitate removal of the ruptured balloon from the bone cavity, the balloon may be treated with special coating chemicals or substances or may be textured to prevent the balloon from sticking to the filler material or cavity walls. In one embodiment, the balloon might open at the distal end. This configuration may allow the balloon to be more readily removed from the bone cavity after the balloon has opened or ruptured.

Also, biologically resorbable balloons may be designed and configured according to the present invention. For instance, a deployed balloon comprising bio-resorbable polymers might be transformed by physiological conditions into substances which are non-harmful and biologically compatible or naturally occurring in the body. These substances may remain in the patient or be expelled from the body via metabolic activity. In one example, a balloon designed to restore the anatomy of a vertebral body would be placed within a prepared cavity inside the treated vertebra and inflated with a radio-opaque filler material. Immediately after inflation (or after the filler material has partially set), the balloon may be disengaged, separated, or detached from the catheter to remain within the bone. As the balloon resorbs new bone may replace the filler material. Alternatively, the filler material may be converted by biological activity into bone or simply remain in the bone.

As one skilled in the art would readily appreciate a deployed balloon may be designed for partial or complete resorption. For instance, a selectively resorbable balloon may be configured to produce a bio-inert implant, structure, or a configuration comprising a plurality of such entities. For example, a balloon may have a resorbable membrane component and a biologically inert structural reinforcing component. In another example, a balloon designed to be selectively resorbable might form a series of bio-inert segments. These bio-inert segments might provide structural containment, or a reinforcing interface at weakened portions of the cortical bone. The segments may also be designed to cooperate and beneficially dissipate post operative stresses generated at the interface between the restored cortical bone and filler material. The precise nature of the stress reduction may be adapted to a particular anatomy.

An implanted balloon may also be designed such that it can be resorbed only after certain conditions are met. For instance, a balloon designed to provide containment in a particular region of unhealthy or damaged cortical bone may eventually be resorbed following one or more triggering conditions. In one example, the return of normal physiological conditions would trigger the break down of the balloon implant. The triggering condition may involve relative temperature, pH, alkalinity, redox potential, and osmotic pressure conditions between the balloon and surrounding bone or cancellous materials.

In another example, a controlled chemical or radiological exposure would trigger the break down of the balloon. For instance, a chemically triggered resorption may include, without limitation, a physician prescribed medicament or specially designed chemical delivered to the balloon via oral ingestion or intravenous injection. An electrical charge or current, exposure to high frequency sound, or X-rays may also be used to trigger biological resorption of the balloon.

Resorbable balloons may also provide an implanted balloon with beneficial non structural properties. For instance, soluble compounds contained within a bio-resorbable sheath may have particular clinical benefits. For example, a resorable balloon may break down when healthy cancellous bone remains in contact with the balloon for about six weeks. The breakdown of the balloon may then expose a medicament placed within the balloon structure as an internal coating. Also, the medicament may be incorporated into the balloon matrix itself to provide a time release function for delivering the medicament. The medicament may promote additional bone growth, generally, or in a particular area. Examples of other such complementary benefits include, without limitation, antibacterial effects that prevent infection and agents that promote muscle, nerve, or cartilaginous regeneration.

In use, the balloon 30 is inserted into a bone cavity that has been prepared to allow the balloon to be placed near the damaged cortical bone. Preferably, the cancellous tissue and bone marrow inside the bone and in the area to be treated may be cleared from the region in advance of deploying the balloon. Clearing the treated region may be accomplished by either shifting or relocating the cancellous bone and marrow to untreated regions inside the bone, or alternatively by removing the materials from the bone. Alternatively, cancellous bone and marrow may be cleared with a reamer, or some other device.

Additionally, the bone cavity may be irrigated and/or aspirated. Preferably, the aspiration would be sufficient to remove bone marrow within the region to be restored. In particular, a region as big as the fully deployed balloon should be aspirated in this manner. More preferably, a region exceeding the extent of the fully deployed balloon by about 2 mm to 4 mm would be aspirated in this manner. Clearing the cavity of substantially all bone marrow near or within the treated region may prove especially useful for restoring the bone and incorporating the balloon as a prosthetic device to remain in the cavity.

Clearing substantially all bone marrow from the treated area also may provide better implant synthesis with the cortical bone, and prevent uncontrolled displacement of bone marrow out of areas of damaged cortical bone. For example, a balloon for restoring a vertebral body may further comprise a prosthetic implant which will remain in the restored vertebrae for an extended period of time. Removing substantially all the bone marrow from the region of the vertebrae to be restored might provide better surface contact between the restored bone and the implant.

One skilled in the art would readily appreciate the clinical benefits for preventing the release of marrow or bone filling material to the vascular system or the spinal canal.

For example, removing substantially all the bone marrow from the treated region of the bone may reduce the potential for inadvertent and systemic damage caused by embolization of foreign materials released to the vascular system. For vertebral bodies, removing the bone marrow may also reduce the potential for damaging the spinal cord from uncontrolled displacement during deployment of the balloon or a subsequent compression of the vertebrae and implant mass.

Further, the cavity may be treated with a sealant to help prevent or reduce leakage of filler material from the cavity or to help prevent bone materials or body fluids from leaching into the cavity. Generally, sealants comprising fibrin or other suitable natural or synthetic constituents may be used for this purpose. The sealant may be applied at any suitable time or way, such as by spray application, irrigation, flushing, topical application. For example, the sealant may be spray coated inside the cavity prior to or after deployment of the balloon. In addition, the sealant may be applied to the balloon exterior as a coating so that the sealant would be delivered to the cavity as the balloon is deployed.

In another example, the sealant may be placed inside the treated area first, and then an inflatable device may be used to push the sealant outward toward the cavity walls. The inflatable device may be rotated or moved axially in order to apply the sealant. Also, the balloon may not be fully pressurized or may be gradually pressurized while the sealant is being applied.

The viscosity or other properties of the sealant may be varied according to the type of delivery and the procedure used. For example, it is preferred that the sealant is a gel if it is placed inside the cavity and the balloon is used to apply it to the cavity walls. As previously described, each of these optional steps regarding the use of a sealant may be performed after inflation of the balloon, or before, or not at all.

Thereafter, the balloon 30 is inserted into the prepared cavity, where it is inflated by fluid, (e.g., saline or a radiopaque solution) under precise pressure control. Preferably, the balloon 30 is inflated directly against the cortical bone to be restored, by an inflation device 15. In this manner, the deployed balloon presses the damaged cortical bone into a configuration that reduces fractures and restores the anatomy of the damaged cortical bone.

Following fracture reduction, the balloon is deflated by releasing the inflation pressure from the apparatus. Preferably, the balloon may be further collapsed by applying negative pressure to the balloon by using a suction syringe. The suction syringe may be the inflation device itself, or an additional syringe, or any other device suitable for deflating the balloon. After the balloon is sufficiently deflated, the balloon may be removed from the cavity, and the bone cavity may be irrigated or aspirated. Optionally, the cavity also may be treated with a sealant. The cavity then can be filled with bone filler material. The bone filler material may be natural or synthetic bone filling material or any other suitable bone cement. As previously described, each of these optional steps may be performed after inflation of the balloon, or before, or not at all.

As described more fully below, the timing of the deflation of the balloon and the filling of the cavity with bone filler material may be varied. In addition, the balloon may not be deflated prior to completing the surgical procedure. Instead, it may remain inside the bone cavity for an extended period. Thus, the method of the present invention relates to creating a cavity in cancellous bone, reducing fractures in damaged cortical bone with a medical balloon, restoring the natural anatomy of the damaged bone, and filling the restored structure of the bone with filling material.

The inflatable device may also be adapted to serve as a prosthetic device outside of a bone. One example is that the balloon may be used as an artificial disk located between two adjacent vertebrae. The use of an inflatable device in this manner may allow for replacement of the nucleus of the treated disk, or alternatively may be used for full replacement of the treated disk. Portions of the treated disk may be removed prior to deploying the inflatable device. The amount of disk material removed may depend upon the condition of the treated disk and the degree to which the treated disk will be replaced or supported by the inflatable device. The treated disk may be entirely removed, for instance, when the inflatable device serves as a complete disk replacement. If the inflatable device will serve to support or replace the nucleus or other portion of the treated disk, then less material, if any, may need to be removed prior to deployment.

The construction and shape of the inflatable device may vary according to its intended use as either a full disk replacement or a nuclear replacement. For instance, an inflatable device intended to fully replace a treated disk may have a thicker balloon membrane or have coatings or other treatments that closely replicate the anatomic structure of a natural disk. Some features include coatings or textures on the outer surface of the inflatable device that help anchor it or bond it to the vertebral endplates that interface with the artificial disk. The balloon membrane also may be configured to replicate the toughness, mechanical behavior, and anatomy of the annulus of a natural disk. The filler material likewise may be tailored to resemble the mechanical behavior of a natural disk.

In another example, if the inflatable device is intended to treat only the nucleus of the disk, the balloon may be designed with a thin wall membrane that conforms to the interior of the natural disk structures that remain intact. In addition, the balloon membrane may be resorbable so that the filler material remains after the inflatable device has been deployed. Alternatively, the balloon membrane may be designed and configured to allow the balloon to be lysed and removed from the patient during surgery. One advantage of this design would be that the balloon may function as a delivery device that allows interoperative measurement of the volume of the filler material introduced into the patient. In addition, this design allows for interoperative adjustment of the volume, so that filler material can be added or removed according to the patient's anatomy before permanent deployment. Other design features of the inflatable device and filler material described herein for other embodiments or uses also may be utilized when designing a balloon as an artificial disk.

In one embodiment of an artificial disk, the balloon is inflated with a radio-opaque material to restore the natural spacing and alignment of the vertebrae. The inflating solution or material may be cured or reacted to form a viscous liquid or deformable and elastic solid. Preferably, such a balloon may comprise an implant possessing material and mechanical properties which approximate a natural and healthy disk. For instance, the balloon may be designed for long term resistance to puncture and rupture damage, and the filler material may be designed and configured to provide pliable, elastic, or fluid like properties. Generally, filler material for a replacement disk balloon may comprise any suitable substance, including synthetic and bio-degradable polymers, hydrogels, and elastomers. For example, a balloon may be partially filled with a hydrogel that is capable of absorbing large volumes of liquid and undergoing reversible swelling. A hydrogel filled balloon may also have a porous or selectively porous containment membrane which allows fluid to move in and out of the balloon as it compressed or expanded. The filler material may also be designed and configured to form a composite structure comprising a solid mass of materials.

Balloons of the present invention also may be adapted for use as a distraction instrument and an implant for interbody fusion, such as for the lumbar or cervical regions. For instance, a inflatable device of the present invention may be used for posterior lumbar interbody fusion (PLIF). A laminotomy, for example, may be performed to expose a window to the operation site comprising a disc space. The disc and the superficial layers of adjacent cartilaginous endplates may then be removed to expose bleeding bone in preparation for receiving a pair of PLIF spacers. A balloon of the present invention may then be inserted into the disk space and inflated to distract the vertebrae. The controlled inflation of the balloon may ensure optimum distraction of the vertebrae and facilitate maximum implant height and neural foraminal decompression. Fluoroscopy and a radio-opaque balloon inflation fluid may assist in determining when a segment is fully distracted.

If the balloon is to serve as a distraction instrument, a bone or synthetic allograft along with cancellous bone graft or filler material may then be implanted into contralateral disc space. Once the implant and other materials are in the desired position, the balloon may be deflated and removed from the disk space and a second implant of the same height may be inserted into that space.

If the balloon is to serve as a spacer for intervertebral body fusion, the balloon may be inflated with a filler material that sets to form an synthetic allograft implant in vivo. Once the implant has been adequately formed, the balloon may be lysed and removed from the disk space. In another example, the inflated balloon is left intact and is separated from the catheter to remain within the disk space as a scaffold for new bone growth. As previously described, a balloon implant also may be resorbed by physiological conditions and expelled from the patient or transformed and remodeled into new bone growth.

For techniques involving multiple deployments of balloons or filler material, different radiographic signatures may be used for each deployment to enhance the quality of fluroscopic imagery and to assist the surgeon in interpreting spacial relationships within the operation site. The use of different radiographic signatures may be used, for example, with inflatable devices when they are used as instruments (such as a bone restoration tool or as a distraction device), when they are used to deliver bone filler material, or when they are used as implants. Additionally, the use of different radiographic signatures may be utilized for multiple deployment of filler material. For instance, a technique involving the deployment of two balloons between adjacent vertebrae might benefit from such an approach. Similarly, other orthopedic procedures, such as vertebroplasty, also may involve the deployment of multiple balloons having different radiographic signatures. In another example, when the balloon of the present invention is used as a PLIF spacer, the filler material within the first of two intervertebral spacer implant balloons may be provided with less radio-opacity then the second implant. As one skilled in the art would readily appreciate, varying the radio-opacity of the respective implants would facilitate fluoroscopic monitoring and deployment of the second implant. In particular, this would prevent a deployed implant on a first side from blocking the fluoroscopic image of a second implant. This advantage may also be realized when differing radiographic signatures are used in any situation involving multiple deployments, such as for multiple deployments of balloons or filler materials as described above.

The radio-opacity of each implant may be varied by incorporating different concentrations of a radio-opaque material within the filler material which inflates the balloon. For example, filler materials comprising two different concentrations of barium sulfate may be used. Similarly, different radio-opaque materials having distinguishable flouroscopic characteristics may be used.

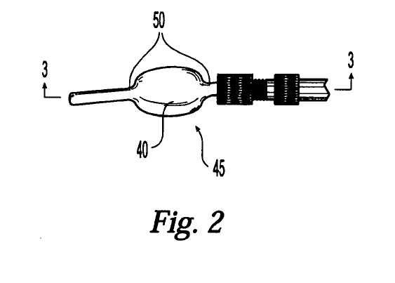

FIG. 2, shows a medical balloon 40 of the construction described above inflated to approximately 200 psi. Preferably, the balloon 40 is made from a single layer of polyurethane material. Multiple balloon layers, and coatings of other materials such as silicone may also be used. For example, the silky texture of an outer silicone layer or coating may be used to facilitate insertion of the balloon 40 or to achieve another clinical objective. One skilled in the related art would recognize that additional materials, layers, and coatings, and combinations thereof, may be used to improve the serviceability of the balloon 40, for example, by increasing the ability of the inflated vessel to resist puncture and tearing. Preferably, the single wall thickness of the balloon 40 may range from approximately about 1.5 mils to about 2.5 mils. A single wall thickness, however, ranging from about 0.5 mil to about 3.5 mils also may be preferred for particular applications. The thickness of optional layers or coatings preferably may range from approximately about 0 mils to about 4 mils. Additionally, radio-opaque indicia (not shown) may be applied to the exterior surface of the balloon 40 to provide an enhanced visual means for assessing the degree of inflation and collapse.

A composite balloon comprising at least two materials that may serve as a reinforcing component and as a boundary forming component. The boundary forming component may be any suitable material used for forming a balloon. Examples of such materials are described more fully herein. The reinforcing component may provide added tensile strength to the balloon by picking up tensile stress normally applied to the boundary forming component of the balloon. The reinforcing component may be designed and configured to distribute these forces evenly about its structure, or may be designed and configured to form a space frame for the deployed balloon structure. The reinforcing component may facilitate better shape control for the balloon and provide for a thinner boundary forming component.

In one embodiment the reinforcing member component may be a braided matrix extending over selected areas of the balloon. In another embodiment, the braided matrix may enclose the balloon structure in its entirety. In another embodiment, braided matrix is on the inside of the boundary forming component of the balloon. Conversely, in another embodiment the braided matrix is located on the outside of the boundary forming component of the balloon. In one embodiment, the braided matrix is located within the boundary forming component. For example, a boundary forming component comprising a membrane might include a braided matrix within the membrane. The reinforcing strength of the braided matrix may be influenced by the type of material from which it is constructed, or by the shape and dimension of the individually braided reinforcing members.

Additionally, the reinforcing strength of the braided matrix may be determined by the tightness of the weave. For example, a more dense pattern for the braided matrix might provide greater strength but less flexability, than a less dense weave of a similar pattern. Also, different patterns may have different combinations of physical characteristics. The angle of the intersecting braided members may also be varied to optimize the physical properties of the balloon. The braided matrix may therefore be customized to provide a certain combination of physical or chemical properties. These properties may include tensile and compressive strength, puncture resistance, chemical inertness, shape control, elasticity, flexability, collapsability, and ability to maintain high levels of performance over the long term. The braided materials may be comprised of any suitable material including nitinol, polyethylene, polyurethane, nylon, natural fibers (e.g., cotton), or synthetic fibers. One firm which manufactures braided matrices of the type described above is Zynergy Core Technology.

As noted above the boundary forming component may comprise a synthetic membrane formed from polyurethane or other materials as described for the general balloon construction. The membrane may be coated on the exterior to enhance non-reactive properties between the balloon and the body, or to ensure that a balloon will not become bonded to the balloon inflation materials. Thus, a lysed balloon may be withdrawn without significant disturbance to the filled cavity. It is expected that a balloon formed from a membrane and braided matrix may designed to operate at an internal pressure of about 300 psi.

As previously described, the size and configuration of the inflation device may vary according to the particular bone to be restored. FIG. 3 illustrates a general construction of a balloon of the present invention. The features described in FIG. 3. include: D1 (the outer diameter of the balloon tubing); D2 (the outer diameter of the working body of the balloon); L1 (the length of the balloon); L2 (the working length of the balloon 70); α (the tapered angle of the balloon's proximal end); and β (the angle of the balloon's distal end). Angles α and β are measured from the longitudinal axis 80 of the balloon. Table 1 presents preferred values for the features of the balloon construction depicted in FIG. 3, as they may apply to particular bone anatomies. Values presented in range 1 represent generally preferred dimensions and characteristics. Values presented in range 2, by comparison, represent more preferred criteria.

TABLE 1
PREFERRED AXIAL BALLOON EMBODIMENTS

| Target Bone | Preferred | D1 | D2 | L1 | L2 | α | β |
|---|---|---|---|---|---|---|---|
| Anatomy | Size | (mm) | (mm) | (mm) | (mm) | (deg.) | (deg.) |
| Vertebral | Range 1 | 1.0-3.5 | 5-30 | 10-35 | 5-25 | 25-80 | 50-105 |
| Body | Range 2 | 1.5-3.0 | 8-26 | 15-25 | 12-20 | 45-65 | 60-86 |
| Distal Radius | Range 1 | 1.0-3.5 | 5-25 | 10-45 | 6-40 | 25-80 | 50-105 |
| | Range 2 | 1.5-3.0 | 8-14 | 15-25 | 12-22 | 45-65 | 60-86 |
| Calcaneus | Range 1 | 1.0-3.5 | 5-25 | 5-35 | 3-33 | 25-80 | 50-105 |
| | Range 2 | 1.5-3.0 | 8-12 | 8-12 | 6-13 | 30-50 | 55-80 |
| Tibial Plateau | Range 1 | 1.0-3.5 | 5-40 | 15-60 | 11-56 | 25-80 | 50-105 |
| | Range 2 | 1.5-3.0 | 12-30 | 20-40 | 16-36 | 45-65 | 60-86 |

As described in Table 1, a preferred balloon for a vertebral body would have tubing 60 with outer diameter D1 that ranges from about 1.5 mm to about 3.0 mm. The tubing 60 preferably would also be suitable for attachment to a 16 gauge catheter. As best shown in FIG. 3, the balloon tip 65 may be sized according to the catheter requirements. Additionally, outer diameter D2 would preferably range from about 8 mm to about 26 mm, and more preferably would be between about 12 mm and about 20 mm. Similarly, the proximal end 75 of the balloon 70 may taper at an approximately uniform angle α from the longitudinal axis 80 of the balloon 70. Preferably, angle α ranges from about 25 degrees to about 80 degrees, and more preferably ranges from about 45 to 60 degrees. The distal end 85 of the balloon 70 may also taper at an approximately uniform angle β from the longitudinal axis 80 of the balloon 70. Preferably, the angle β ranges from about 90 degrees to about 50 degrees, and more preferably ranges from about 60 to 86. Further, length L1 of the balloon 70, preferably ranges from about 15 mm to about 30 mm, and the working length L2 of the balloon 70 preferably ranges from about 10 mm to about 20 mm. More preferably, however, length L1 of the balloon 70 ranges from about 20 mm to 25 mm, and the working length L2 of the balloon 70 ranges from about 12 mm to about 15 mm.

The preferred embodiments described above include preferred sizes and shapes for balloons comprising a braided matrix and membrane. As previously noted such a balloon may be adapted to remain with a vertebral body, as a prosthetic device or implant.

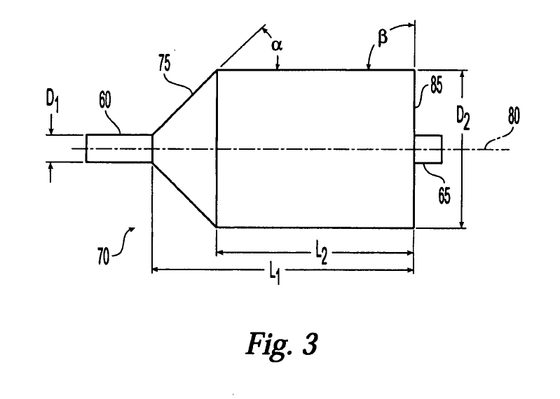

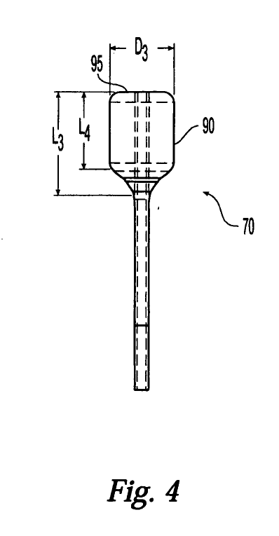

FIGS. 4-6 show preferred embodiments of the axial balloon 70 described in FIG. 3 and Table 1. Although, the following discussion is directed toward exemplary balloon embodiments for deployment in vertebral bodies, these balloons may be used in any suitable bone. Thus, the dimensions and configurations of the balloon styles described in this figures may be varied to accommodate the type of bone or cavity in which the balloon is to be deployed.

FIG. 4 depicts a balloon embodiment style with an uniform bulge 90 having an axially uniform diameter D3 with a blunt distal end 95. In one embodiment, the total length L3 is about 20 mm, the working length L4 is about 15 mm, and the outer diameter D3 is about 12 mm. In another embodiment, the total length L3 is about 20 mm, the working length L4 is about 15 mm, and the outer diameter D3 is about 8 mm. In yet another embodiment, the total length L3 is about 15 mm, the working length L4 is about 10 mm, and the outer diameter D3 is about 8 mm.

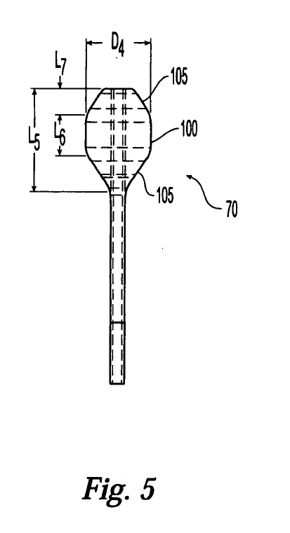

FIG. 5 depicts a balloon embodiment style with a central bulge 100 having a constant outer diameter D4 in a central portion of the balloon 70, while having uniformly tapered ends 105. In one embodiment, a balloon with a central bulge has a total length L5 of about 20 mm, a working length L6 of about 8 mm, a horizontal length L7 of the tapered distal end of about 5 mm, and an overall outer diameter D4 of about 12 mm. In another embodiment, the total length L5 is about 20 mm, the working length L4 is about 8 mm, the horizontal length L7 of the tapered distal end is about 5 mm, and the overall outer diameter D4 of the balloon is about 8 mm. In another embodiment, the total length L5 is about 15 mm, the working length L4 is about 8 mm, the horizontal length L7 of the tapered distal end is about 5 mm, and the overall outer diameter D4 of the balloon is about 8 mm. One skilled in the art would appreciate that the tapered end of this balloon style may have other configurations. For instance, the balloon may have a series of uniform tapered lengths, rather than a single uniform tapered end. Also, the balloon may have a curved tapered end, rather than one or more uniform tapered lengths.

Similarly, the balloon may have a combination of uniform and curved lengths comprising the tapered end of the balloon. The tapered end also may be unsymmetrical about the central axis of the balloon. A balloon comprising a braided matrix and membrane components may be of particular use in developing balloons having a tapered end or unsymmetrical geometry because the braided material can be used to improve shape control or create a space frame for the deployed balloon.

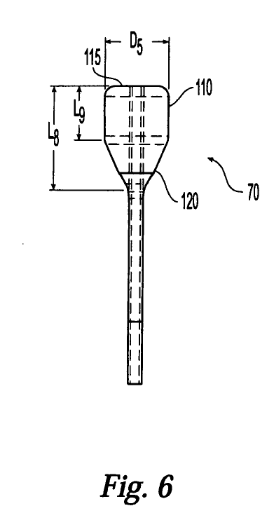

FIG. 6 depicts a balloon embodiment style with an distal bulge 110 having a constant outer diameter D5 in a region abutting a blunt distal end 115, and a uniformly tapered proximal end 120. In one embodiment, the total length L8 is about 20 mm, the working length L9 is about 8 mm, and the outer diameter D5 is about 12 mm. In another embodiment, the total length L8 is about 20 mm, the working length L9 is about 8 mm, and the outer diameter D5 is about 8 mm. In yet another embodiment, the total length L8 is about 15 mm, the working length L9 is about 8 mm, and the outer diameter D5 is about 8 mm. As previously described, one skilled in the art would appreciate that the tapered end of this balloon style may have other configurations. Further, the surprising advantages of the balloon styles depicted in FIGS. 4-6 may be achieved by using a curved or bent catheter.

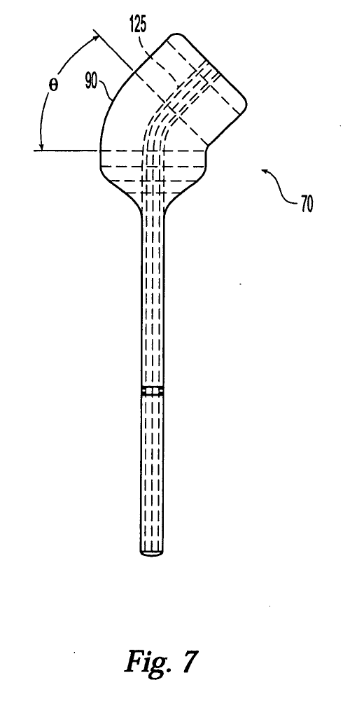

FIG. 7 depicts an exemplary embodiment of the balloon of FIG. 4 having a bend of angle θ along its working length. One skilled in the art would appreciate that more than one bend in the catheter may be used to provide further surprising advantages to the device. Similarly, the catheter may be constructed from a shape memory metal so that the balloon may positioned or deployed in one configuration and then repositioned or deployed in a second configuration at the selective control of the surgeon. Thus, a balloon may be configured for optimal positioning, deployment, and removal from the target cavity. For instance, balloons fitted to shape memory catheters may be deployed to restore the natural anatomy of right and left bones, or the left and right sides of bones with a sagittal plane of symmetry. Preferably, as shown in FIG. 7 the bend of angle θ is obtuse. In another embodiment, the balloon catheter may be incorporate a number of successive bends to create a balloon with parallel central axes.

Similarly, the balloon styles depicted in FIGS. 4-6 and the more general balloon configurations defined by FIG. 3 and Table 1 may be angled from the central catheter.

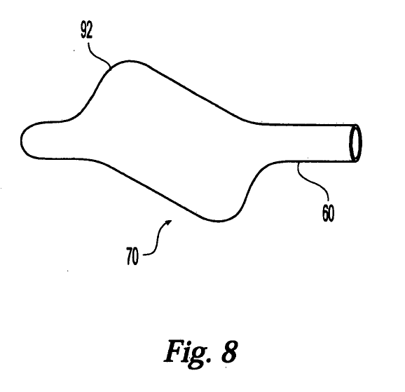

FIG. 8, depicts an exemplary embodiment of balloon 70 with an angled uniform bulge 92. Angle δ, preferably, is acute. One skilled in the art would appreciate that balloons shaped for particular bone cavities or with additional surprising advantages may be developed by using an angled or curved catheter made from shape memory metal as previously described.

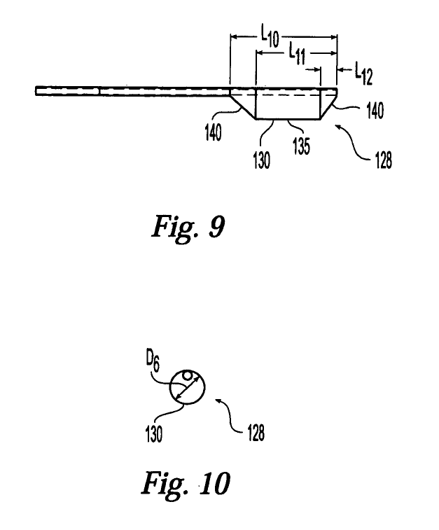

Referring to FIGS. 9-16, preferred balloon configurations may also be developed from offset balloons, including constructions with curved or angled catheters. FIGS. 9-12 depict general embodiments of an exemplary offset balloon.

FIGS. 9 and 10 show an embodiment style of a balloon 128, which is characterized by an offset balloon 130 having an uniform circular bulge 135 in the center of the balloon 130 and uniformly tapering ends 140. The total length L10 of the balloon 130 is divided into a proximal tapered end, a central working section having uniform outer diameter D6, and a distal tapered end. The horizontal length of each of these sections may be defined with respect to the distal end of the balloon. For example, length L11 represents the horizontal distance of the distal tapered end plus the length of the central working section.

Length L12 represents the horizontal length of the distal tapered end. Table 2 presents general preferred and preferred size ranges for this balloon configuration by target bone anatomy. Values presented in range 1 represent generally preferred dimensions and characteristics. Values presented in range 2, by comparison, represent more preferred criteria.

TABLE 2
PREFERRED EMBODIMENTS FOR OFFSET BALLOONS
WITH CIRCULAR CROSS-SECTION

| | | D6 | L10 | L11(a) | L12 |
|---|---|---|---|---|---|
| Target geometry | Preferred Size | (mm) | (mm) | (mm) | (mm) |
| Vertebral Body | Range 1 | 5-30 | 10-35 | 8-25 | 0-5 |
| | Range 2 | 6-20 | 15-25 | 12-22 | 1-3 |
| Distal Radius | Range 1 | 5-25 | 10-45 | 6-40 | 0-5 |
| | Range 2 | 8-14 | 15-25 | 12-22 | 1-3 |
| Calcaneus | Range 1 | 5-25 | 5-35 | 3-33 | 0-5 |
| | Range 2 | 8-12 | 12-28 | 8-24 | 1-3 |
| Tibial Plateau | Range 1 | 5-40 | 15-60 | 11-56 | 0-5 |
| | Range 2 | 12-30 | 20-40 | 16-36 | 1-3 |

(a) Where L11 includes L12

As described in Table 2, the following exemplary embodiments are primarily directed toward vertebral bodies. In one embodiment, the total length L10 is about 20 mm, the working length L11 is about 15 mm, the horizontal distance L12 of the tapered distal end is about 3 mm, and the outer diameter D6 of the circular bulge is about 6 mm. In another embodiment, the balloon has similar dimensions except that the outer diameter D6 is about 8 mm. In yet another embodiment, the balloon diameter D6 is about 12 mm.

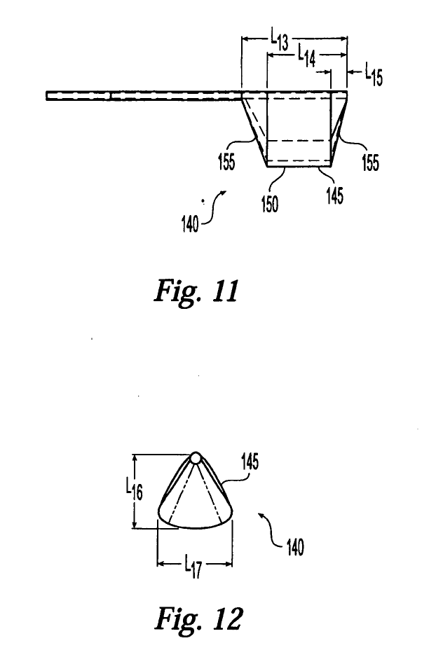

FIGS. 11 and 12, by contrast, show an embodiment style of a balloon 140, which is characterized by an offset balloon 145 having a non-uniform circular bulge 150 in the center of the balloon 145 and uniformly tapering ends 155. The total length L13 of the balloon 140 is divided into a tapered distal end, central working section, and proximal tapered end. The balloon has non uniform cross-section which may be defined by vertical length L16 and cross sectional width L17. Length L14 represents the horizontal distance from the distal end of the balloon. Table 3 presents general and preferred size ranges for this balloon configuration by target bone anatomy. Values presented in range 1 represent generally preferred dimensions and characteristics. Values presented in range 2, by comparison, represent more preferred criteria.

TABLE 3
PREFERRED EMBODIMENTS FOR OFFSET BALLOONS
WITH NON CIRCULAR CROSS-SECTION

| Target | Preferred | L13 | L14 | L15 | L16 | L17 |
|---|---|---|---|---|---|---|
| geometry | Size | (mm) | (mm) | (mm) | (mm) | (mm) |
| Vertebral Body | Range 1 | 10-35 | 8-25 | 0-5 | 5-30 | 5-30 |
| | Range 2 | 15-25 | 12-22 | 1-3 | 6-20 | 6-20 |
| Distal Radius | Range 1 | 10-45 | 6-40 | 0-5 | 5-25 | 5-25 |
| | Range 2 | 15-25 | 12-22 | 1-3 | 8-14 | 8-14 |
| Calcaneus | Range 1 | 5-35 | 3-33 | 0-5 | 5-25 | 5-25 |
| | Range 2 | 12-28 | 8-24 | 1-3 | 8-12 | 8-12 |
| Tibial Plateau | Range 1 | 15-60 | 11-50 | 0-5 | 5-40 | 5-40 |
| | Range 2 | 20-40 | 16-36 | 1-3 | 12-30 | 12-30 |

As described in Table 3, the following exemplary embodiment is primarily directed toward vertebral bodies. In one embodiment, the total length L13 is about 20 mm, the working length L14 is about 15 mm, and the horizontal distance L15 of the tapered distal end is about 3 mm. Further, the vertical height L16 and the lateral width L17 of the balloon 145 are 14 mm and 14 mm, respectively.

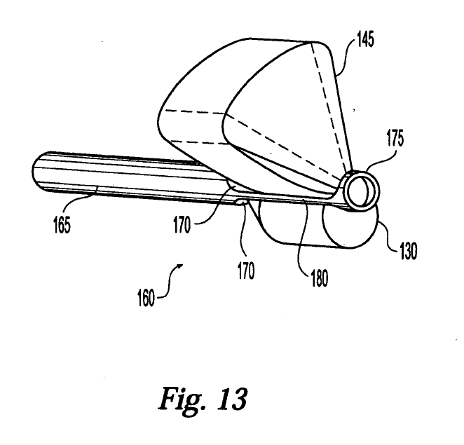

Referring to FIGS. 9 and 12, these general balloon embodiments and the preferred dimensions presented in Tables 2 and 3 may be combined to create complex balloons, which are inflatable structures comprised of a plurality of balloons. For instance, FIG. 13 depicts an embodiment of a complex balloon 160, and a catheter 165 adapted to deploy two inflatable structures 130 and 145. In this exemplary embodiment, the balloons 130 and 145 which comprise the complex inflatable structure 165 are fully seated within the catheter 160, and are deployed through openings 170 around the catheter 165 circumference.

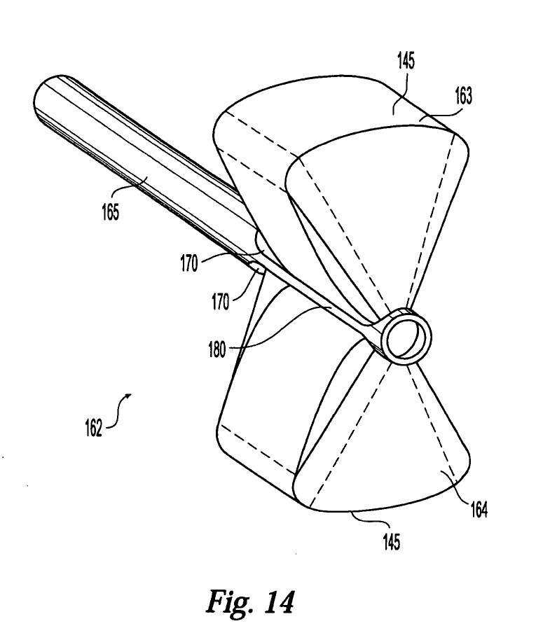

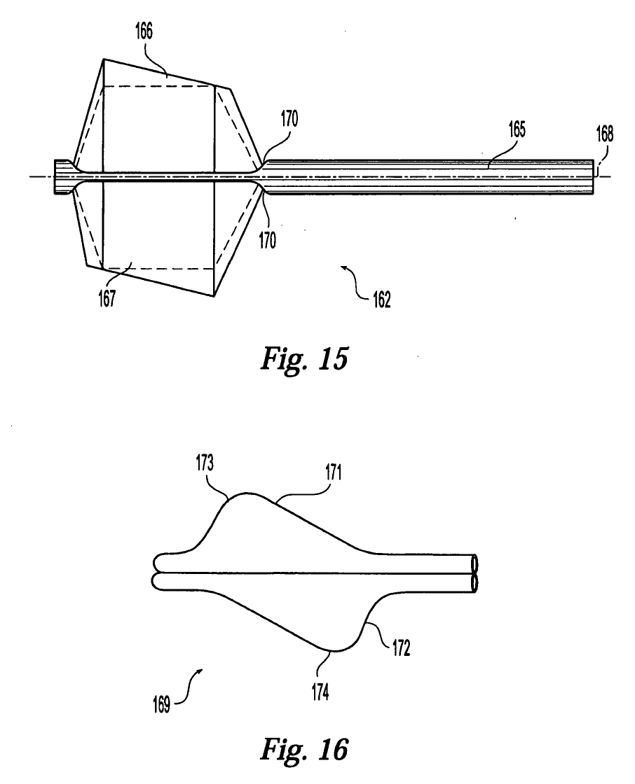

FIG. 14 depicts an embodiment of a single balloon 162 with two chambers 163 and 164 each of which are shaped like offset balloon style 145. By contrast, FIG. 15 depicts another embodiment of the balloon of FIG. 14, wherein the balloon chambers 166 and 167 are angled with respect to the longitudinal axis 168 of the balloon catheter 165.

Additionally, in other general embodiments of complex balloons as depicted in FIG. 14, balloons with circular cross-sections, or other suitable geometric shapes may be used.

In yet another exemplary embodiment of a complex balloon, FIG. 16 shows balloon 169 comprising two individual balloons 171 and 172, each having a tapered bulge 173 and 174 that produce a complex and angled embodiment of the balloon of FIG. 14. As the foregoing discussion suggests, complex balloons may be constructed for particular bone geometries or clinical purposes. One skilled in the art would appreciate that angled balloons such as those depicted in FIGS. 15 and 16 can be made for an anatomically correct fit, as previously described, without requiring an angled catheter shaft. One skilled in the art would further appreciate the potential reduction in cost an angled balloon construction might posses over a similarly shaped angled catheter balloon.

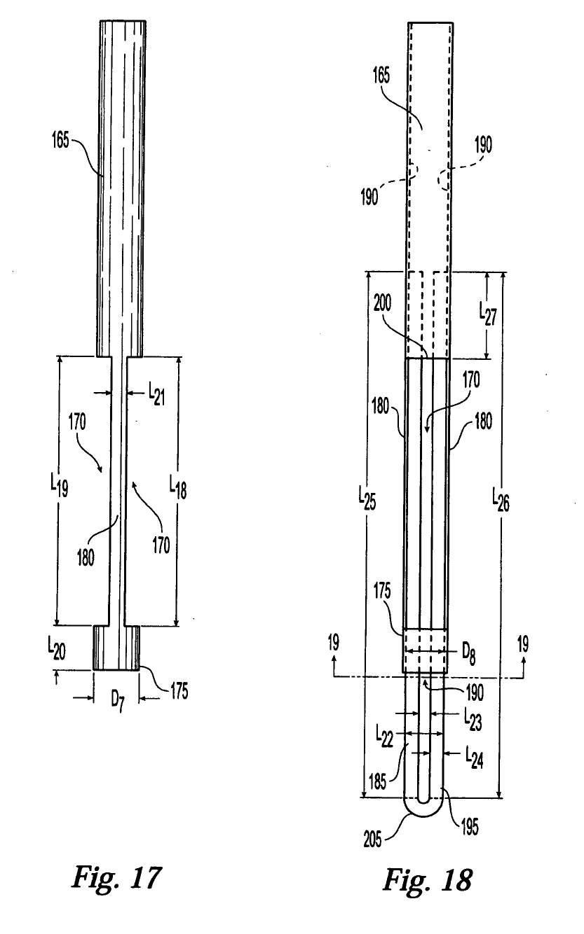

FIGS. 17-25 depict exemplary embodiments of a catheter construction of the present invention. The basic components of the catheter are shown in FIGS. 17-19. Additional, illustrative embodiments of structural reinforcing elements are presented in FIGS. 20-25. In general the catheter may be constructed with a plurality of openings through which a balloon or plurality of balloons may be deployed. For example, the catheter may have two openings through which a single balloon may be deployed. As the balloon is inflated, the reinforcing members of the catheter that define the openings cause the balloon to expand outwardly away from the catheter. Alternatively, a plurality of balloons may be deployed through the windows either at approximately the same time or in a staged succession. The balloons also may have differing shapes, surface characteristics, or pressures to suit a particular clinical application. The following discussion illustrates non-limiting examples of the present invention using a catheter with windows through which a balloon or balloons are deployed.

FIG. 17 depicts the distal end 175 of the catheter 165 of FIG. 13 in an elevation view. The catheter 165 has an outer diameter D7, a proximal tip length L20, and two circumferentially opposed balloon deployment openings 170. Lengths L18 and L19 of the balloon deployment openings 170, preferably, are the same length. The openings 170, however, may be of different length and size to accommodate a particular balloon. The remaining catheter material 180 between the balloon deployment openings 170 form strips of width L21. Generally, the number of strips 180 correspond to the number of balloon deployment openings 170 provided in the catheter 165. Similarly, the width L21 of each strip 180 may depend on the number of strips 180 provided and the outer diameter D7 of the catheter 165.

FIG. 18 shows the principle structural components of the catheter of FIGS. 17 and 18. The catheter 165 is constructed with inner dimension D8, and an U-rod 185 that is inserted into the catheter 165 via an opening 190 in the distal tip 175. The width L22 of the outer dimension of the U-rod 185 may be sized according to the inner diameter D8, such that the U-rod 185 fits within and bears against the inside wall 190 of the catheter 165. Length L24, the outer dimension of the individual rod 195, is related to the structural reinforcement required for the intermediate catheter strips 180 located between the balloon deployment windows 170. Although, the interior width L23 of the U-rod 185 is related to the geometry of the catheter interior, width L23 is also operably configured to cooperate with the deployed balloon or balloons.

In addition, length L25 and length L26 of the U-rod 185 preferably extend beyond the distal edge 200 of the balloon deployment opening 170 to provide a suitable anchoring length L27 for the U-rod 185 within the catheter 165. U-rod segment lengths L25 and L26 need not be equal. The rounded tip 205 of the U-rod 185 may be fully recessed or may partially extend from the proximal end 175 of the catheter 165. In one embodiment, the tip 205 of the U-rod 185 is secured to the catheter 165 by a soldered, brazed or welded connection. A glued fastener or other attachment means may also be used. For instance, a snap together fastening method may be used. Depending on the number of balloon deployment openings 170 and the material of catheter 165 construction, the number of reinforcing rods 185 will vary. Also, the means for joining a plurality of reinforcing rods 185 together and connecting the reinforcing rods 185 to the catheter 165 may vary from the embodiments shown.

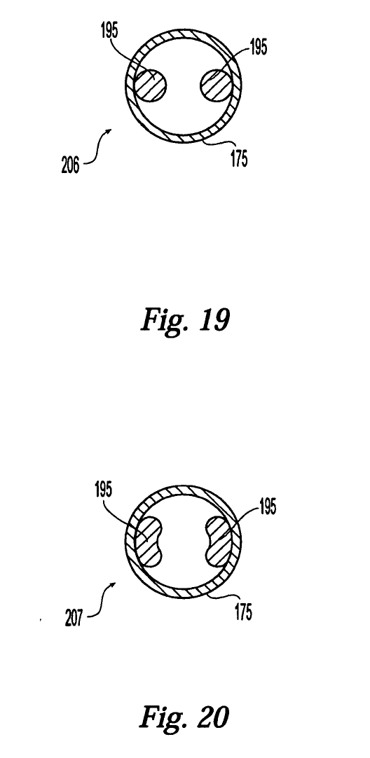

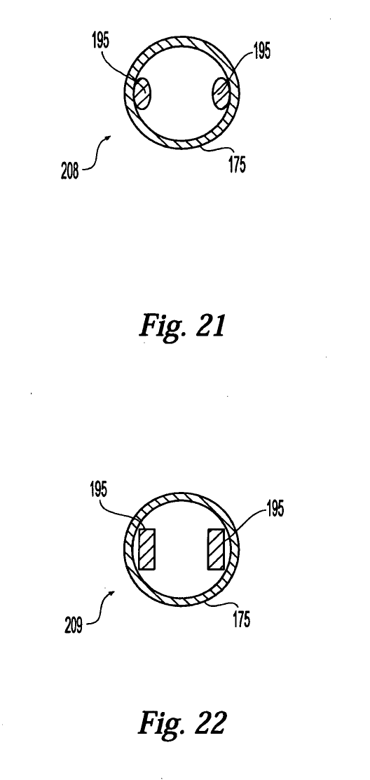

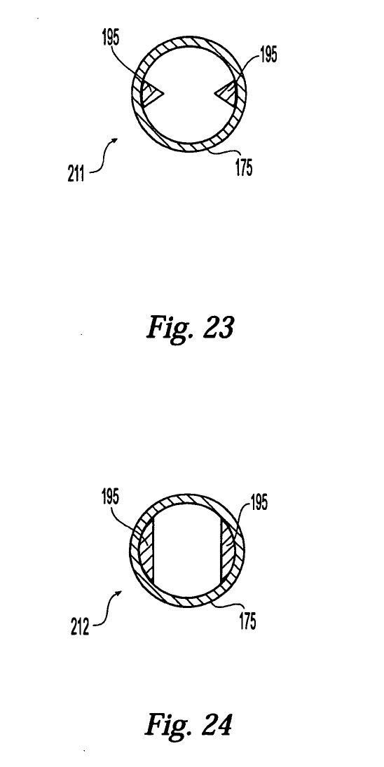

FIG. 19 is a sectional view through line 19-19 of FIG. 18 and shows individual reinforcing rods 195 with an exemplary cross section. In this illustrative embodiment, reinforcing rod 206 is circular in cross-section. As one skilled in the art would appreciate, the geometry of the reinforcing rod may be selected to provide a beneficial combination of clearance and strength. For instance, FIGS. 20-25 depict individual reinforcing rods 195 with other illustrative geometric cross sections. The reinforcing rod of FIG. 20 shows an embodiment with kidney bean shaped cross-section. FIG. 21 shows a reinforcing rod with oval shaped cross-section. FIGS. 22 and 23 show reinforcing rod embodiments with rectangular and triangular shaped cross-sections, respectively. FIG. 24, by contrast, shows an exemplary reinforcing rod with a circular section shaped cross-section.

In addition, multiple rods may be used instead of a U-rod to accommodate a reinforced catheter with a plurality of balloon deployment openings. One skilled in the art would readily appreciate that one particular geometry of reinforcing rods may prove easiest to manufacture, assemble, or configure. Therefore, one embodiment may prove to be the most cost effective solution for a particular balloon configuration. For this reason, these embodiments are not intended to be a complete set of cross sections contemplated by the invention, rather general illustrations of the reinforcing rod concept. TABLE 4 presents general dimensions for the catheter depicted in FIGS. 17-18. Values presented in range 1 represent generally preferred dimensions and characteristics. Values presented in range 2, by comparison, represent more preferred criteria.

TABLE 4
PREFERRED EMBODIMENTS FOR WINDOWED CATHETERS

| | | D7 | D8 | L18, | | | | | | L25 |
|---|---|---|---|---|---|---|---|---|---|---|
| Target Bone | Preferred | (a) | (b) | L19 | L20 | L21 | L22 | L23 | L24 | L26 |
| Anatomy | Size | (mm) | (mm) | (mm) | (mm) | (mm) | (mm) | (mm) | (mm) | (mm) |
| Vertebral | Range 1 | 2-7 | 1.5-6.9 | 10-35 | 0.25-10 | 0.2-4 | 0.5-6.9 | 0.5-6.5 | 0.2-3 | 10.5-50 |
| Body | Range 2 | 3-5 | 2.5-4.9 | 15-25 | 2-6 | 0.5-2.5 | 2-4.9 | 1.5-4 | 0.5-1.75 | 18-35 |
| Distal | Range 1 | 2-7 | 1.5-6.9 | 10-45 | 0.25-10 | 0.2-4 | 0.5-6.9 | 0.5-6.5 | 0.2-3 | 10.5-65 |
| Radius | Range 2 | 3-5 | 2.5-4.9 | 15-25 | 2-6 | 0.5-2.5 | 2-4.9 | 1.5-4 | 0.5-1.75 | 18-35 |
| Calcaneus | Range 1 | 2-7 | 1.5-6.9 | 5-35 | 0.25-10 | 0.2-4 | 0.5-6.9 | 0.5-6.5 | 0.2-3 | 5.5-50 |
| | Range 2 | 3-5 | 2.5-4.9 | 12-28 | 2-6 | 0.5-2.5 | 2-4.9 | 1.5-4 | 0.5-1.75 | 15-35 |
| Tibial | Range 1 | 2-9 | 1.5-8.9 | 15-60 | 0.25-10 | 0.2-6.5 | 0.5-8.9 | 0.5-8.5 | 0.2-4 | 13.5-80 |
| Plateau | Range 2 | 3-7 | 2.5-6.8 | 20-40 | 2-6 | 0.5-4 | 2-6.8 | 1.5-6 | 0.5-2.5 | 18-50 |

(a) Outer Diameter
(b) Inner Diameter

Reinforcing elements, alternatively, may be individual rails which are connected to and oriented around the catheter perimeter by a plurality of spacer rings which are mounted on an internal lumen. The reinforcing elements may further be wire elements that are post tensioned at the distal tip of the catheter. For this reason, the relative sizing of the balloon deployment window, the catheter strips and the reinforcing elements may be reconfigured to accommodate a particular anatomical, mechanical, therapeutic, or clinical need.

For example, FIG. 25 shows an alternative reinforcing structure to the rods depicted in FIGS. 17-24. The reinforcing member of FIG. 25 may be tubular in construction and provided with a slot 213 for deploying one or more inflatable devices. The tubular reinforcing element may formed by a special extrusion that provides, for example, thicker (i.e., stronger) walls in selected locations. As one skilled in the art would appreciate, a tubular catheter reinforcing member may require more than one slot to accommodate a device with a plurality of balloon deployment windows. Additionally, more than one balloon may be deployed through each deployment window. Thus, in one embodiment, a single balloon with a plurality of chambers may be deployed through one deployment window. In another embodiment, two separate balloons may be deployed through a single deployment opening. In yet another embodiment, a single balloon with a plurality of chambers may be deployed through an equal number of balloon deployment openings.

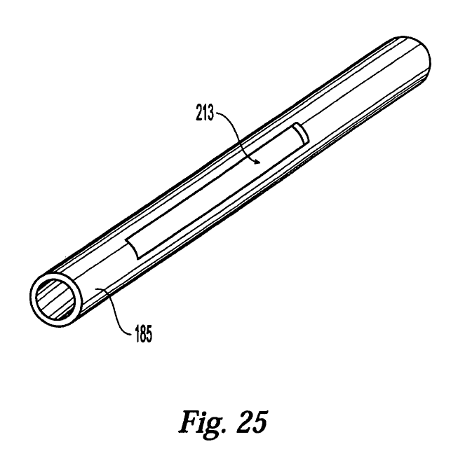

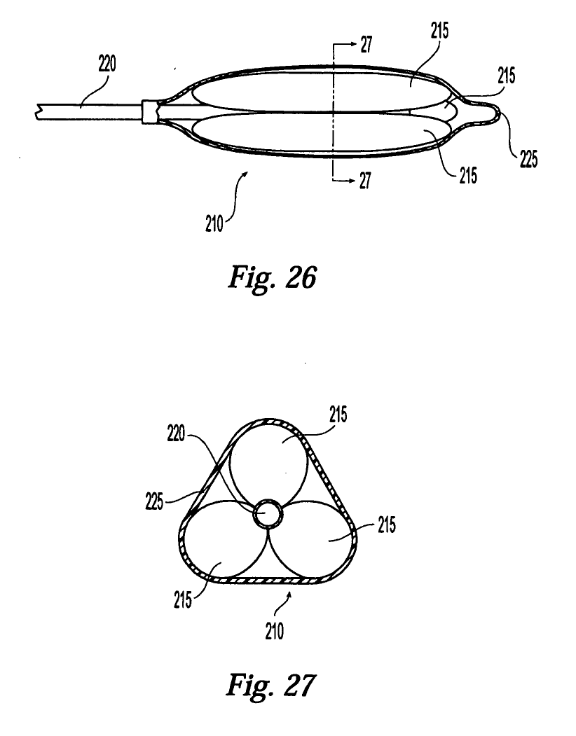

FIGS. 26-35, show illustrative complex balloon embodiments constructed from the balloons described in the fore going figures and tables.

FIGS. 26 and 27 show a balloon catheter with three balloons and three deployment windows. The complex balloon 210 comprises three offset circular balloons 215 stemming from a central catheter 220 and enclosed by an optional outer layer 225. In one embodiment, the individual balloons 215 are comprised of single layers. In another embodiment, the individual balloons may be formed from a plurality of layers and materials. Alternatively, in another embodiment, the complex balloon may comprise a single balloon with three chambers enclosed by an optional outer layer 225. One skilled in the art would readily appreciate that the thickness of each of these layers may be different, and that complex balloons may achieve large effective outer diameters, with thinner balloon walls. Thus, a complex balloon may provide surprising benefits and high levels of clinical performance including: increased resistance to puncture and tearing, novel positioning abilities, enhanced deployment, improved retractability, and ease of removal.

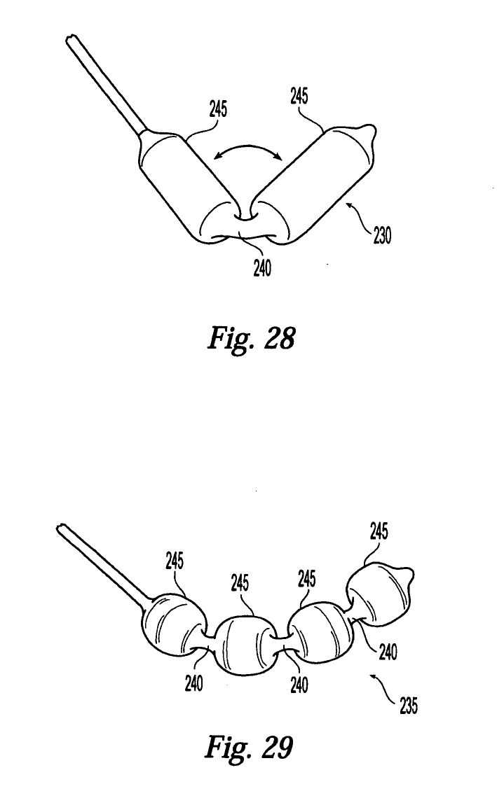

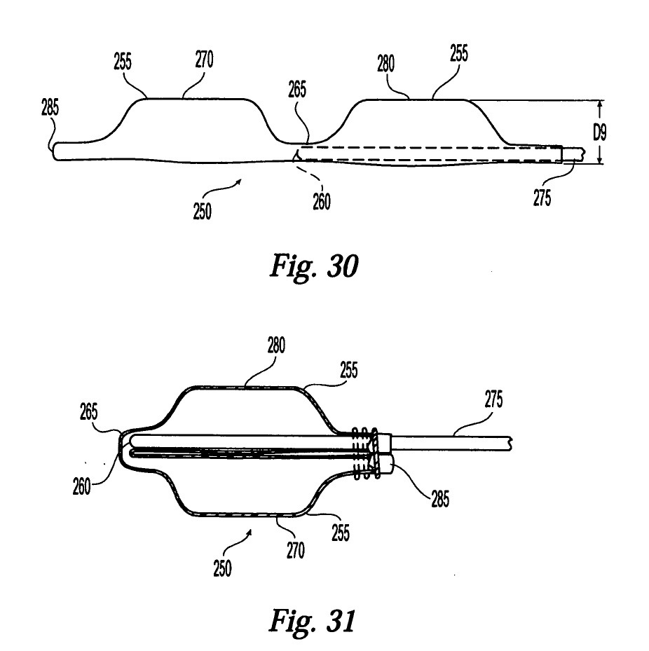

FIGS. 28 and 29, depict axial balloon embodiments 230 and 235 having uniform diameter and at least one integral hinge 240, which separates the working length of the balloon into a plurality of segments. Adjacent balloon segments are free to move about the common hinge. FIG. 30, by contrast, represents an offset balloon 250 with two large chambers 255 connected in serial. In one embodiment, a catheter tip 260 is inserted into the balloon 250 to a point 265 about equidistant from the balloon chambers 255. Referring to FIG. 31, the second chamber 270 of the balloon 250 is then folded over the tip 260 and doubled back along the length of the catheter 275. Further, the folded over portion 270 of the balloon 255 may be secured to the non-folded portion 280 and tied to the catheter 275 near the proximal end of the balloon. In another embodiment, the doubled chambered balloon is constructed of two layers. In yet another embodiment, an inner balloon is folded about the catheter and then the entire composite structure is then wrapped within an additional outer layer. In one embodiment, the outer diameter D9 of the balloon 250 ranges from 2 mm to 12 mm.

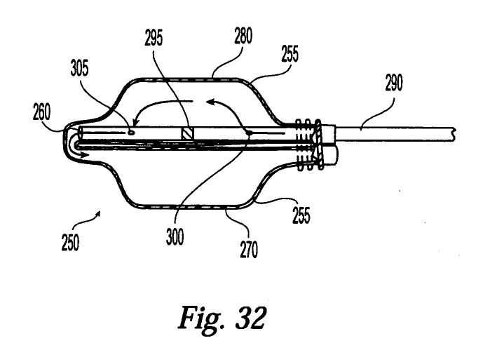

In yet another embodiment, shown in FIG. 32, a specially constructed catheter 290 may be used to provide fluid to the balloon chambers 255 in a sequential manner. During balloon inflation, fluid is prevented from being transported directly into the second chamber 270 of the balloon 250, by a closed valve or blockage 295 in the catheter. The inflation fluid is directed into the first chamber 280 of the balloon 250 via an aperture 300 located on the distal side of the blockage 295 in the catheter 290. The fluid partially fills the first balloon chamber 280, and then renters the catheter 290 via an additional aperture 305 located on the proximal side of the blockage 295 in the catheter 290. As the fluid continues to fill the first chamber 280 of the balloon 250, fluid also starts to migrate through the proximal end of the blocked catheter 290 to the second balloon chamber 270 via an opening in the tip 260 of the catheter 290.

As shown further in FIG. 32, the proximal tip 260 of the catheter 290 provides a fluid connection between the first 280 and second 270 balloon chambers 255. When the balloon 250 is deflated, the direction of fluid transport is reversed. In one embodiment, the blockage 295 in the catheter 290 is removed to allow fluid flow throughout the length of the catheter 290. In another embodiment, a pressure activated valve, opens to permit free fluid flow through the catheter, when the pressure in the second chamber 270 of the balloon 250 becomes larger than a predetermined pressure in the first balloon chamber 280. In yet another embodiment, the catheter blockage 295 may be selectively controlled by the surgeon and formed from a shape memory metal, that would provide by-pass flow in one state, and direct catheter flow in a second state.

One skilled in the art would readily appreciate that more apertures may be used as appropriate to effect the desired rate of fluid transfer, and that a folded multi-chamber balloon may be simple to assemble and test during manufacturing. Thus, creating complex balloons from a folded multi-chamber balloon 250 embodiments may also provide cost savings.

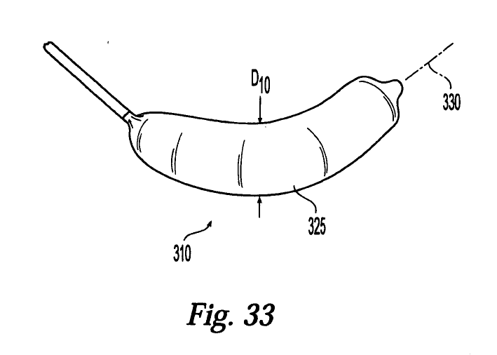

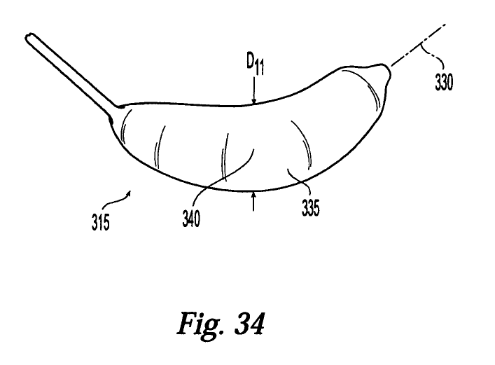

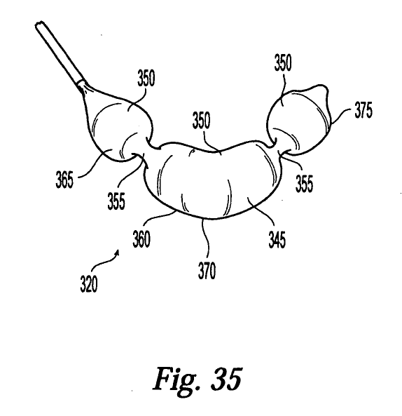

Similarly, FIGS. 33-35 depict additional exemplary balloon embodiments 310, 315, and 320. FIG. 33, for example, depicts an axially offset balloon 325 with a uniform diameter D10 and curved shape. In one embodiment, the curved balloon 325 having longitudinal axis 330 may intimately contact the walls of the prepared bone cavity. Alignment of balloon-applied forces with the bone damage facilitates a shape appropriate restoration of the bone anatomy. In another embodiment, the curve is provided by a curved catheter or a catheter made from shape memory metal, rather, than molding the shape into the balloon. In another embodiment, the curved balloon is formed from an axially offset balloon 335 having non uniform diameter. For example, the balloon of FIG. 34 has a diameter that varies along the longitudinal axis of the balloon. In the illustrative embodiment shown in FIG. 34, the largest diameter D11 is located at the longitudinal 330 mid-point 340 of the balloon 335. In yet another embodiment, shown in FIG. 35, the balloon 320 has three chambers 350, two hinges 355, and a curved central section 365. In another embodiment of the balloon of FIG. 35, the structure of the balloon 320 allows for the controlled inflation and deflation of the individual chambers 365, 370, and 375.

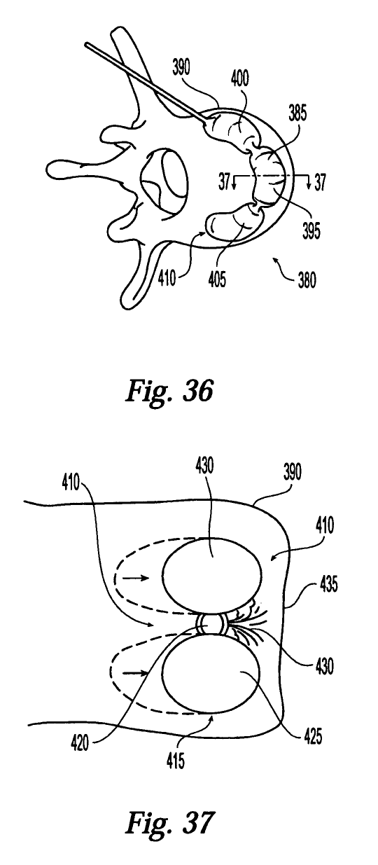

FIG. 36 depicts a sectional view through the longitudinal axis of the spine, and shows a multi-chambered and hinged balloon 385 within a vertebral bone 390. In this embodiment the complex balloon has the advantage of allowing selected chambers (e.g., chamber 395) to be deflated first before the other sections (e.g. chambers 400 and 405). Thus, cavity 410 could then be partially filled with bone cement without deflating or removing the outer balloon chambers 400 and 405 and the restored anatomy of the bone 390 could be fully or nearly fully maintained during the transition from bone fracture reduction to bone fixation. In another embodiment, bone filling material can be applied to the cavity as the balloon sections are deflated. In yet another series of embodiments, the multiple balloon chambers of FIGS. 35-37 may be formed from shared septum membranes, rather, than narrowed passageways or hinges.

FIG. 37 which is taken along line 37-37 of FIG. 36, depicts a central catheter 420 with offset circular balloons 425 and 430. As the top 430 and bottom 425 balloon are deflated, bone cement 430 is filled against the outer wall 435 of the cavity 410 in the restored vertebrae 390. In this fashion, a controlled volume exchange between the inflated structure 430 and 425 and the bone filling material 430 is accomplished. Thus, multiple-chambered balloons offer the potential for surprising advantages, such as controlled volume exchange between the restorative balloon and the bone filling material. Similarly, one skilled in the art would also readily appreciate that multi-chamber balloons may also be used for sequential filling of restored bone cavities. For example, the inflated structure in a stronger part of the bone may be deflated while balloons supporting weaker portions of the bone remain deployed. The region of the bone where the balloon is deflated may then be filled with bone filler material and allowed to harden or gel. Then, neighboring or other balloons may be selectively deflated and the regions filled in a similar manner. In this manner, controlled deflation of a multi-chambered balloon provides temporary support to selected areas of the restored bone anatomy while other areas are filled with bone filler material.

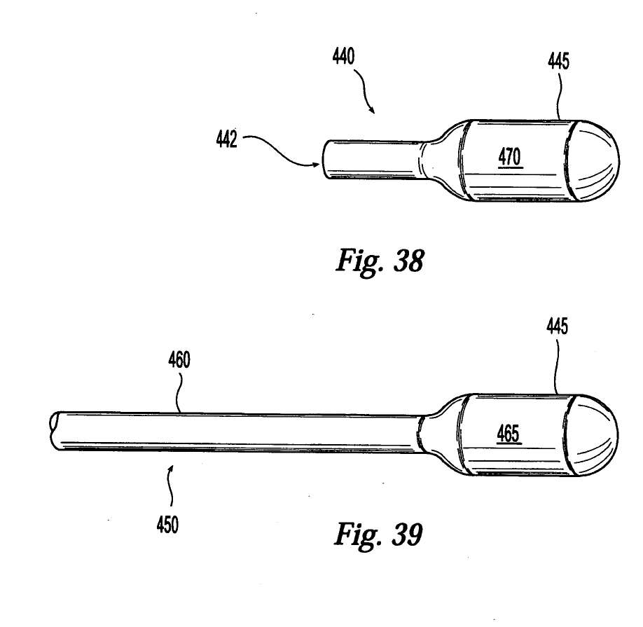

FIG. 38 shows an exemplary embodiment of a balloon or containment device 440 that may be formed of biodegradable materials 445. All, or part of the containment device 440 may be resorbable. Suitable biodegradable materials may comprise non-polymers (e.g., collagen), conventional biodegradable polymer materials (e.g., 70:30 materials with or without plasticizers), or specially formulated polymers for medical applications (e.g., biodegradable polyurethanes).

Non-limiting examples of specially formulated biodegradable polyurethanes are disclosed in the following exemplary published materials, the contents of which is fully incorporated herein by reference: (1) Goma, K., and Gogolewski, S., “In vitro degradation of novel medical biodegradable aliphatic polyurethanes based on e-caprolactone and Pluronics® with various hydrophilicities,” Polymer Degradation and Stability 75 (2002), pp. 113-122; and (2) Gorna, K., and Gogolewski, S., “Novel Biodegradable Polyurethanes for Medical Applications,” Synthetic Bioabsorbable Polymers for Implants, ASTM STP 1396, C. M. Agrawal, J. E. Parr, and S. T. Lin, Eds. American Society for Testing and Materials, West Conshohocken, Pa., 2000.

Nevertheless, the balloon or containment device need not comprise a resorbable material. For example, the containment device may comprise an inherently rigid polyester material that is prepared as a sufficiently thin barrier so as to have the desired flexibility and strength for use as a balloon or containment device. Such a barrier may be formed with or without conventional plasticizers known in the related art.

The containment device may have one or more openings 442 for receiving filler material. The containment device may be used to contain and capture various bone void fillers, such as bone cement, calcium phosphate cement, bone chips, and demineralized bone, within a bone void. The containment device 440 is inserted into a void in the bone, and the containment device is then backfilled with a bone void filler. The bone void filler material may expand the containment device into the available space or the containment device may be placed against the bone void surfaces in a partially or fully expanded position and then filled with bone void filler. The device contains the filler material, preventing extravasation (extraosseous flow) of the filler material into surrounding tissues, and then degrades in vivo. The containment device may be used, for example, in filling bone voids in the vertebral bodies of the spine, as well as in long bones and the craniomaxillofacial skeleton.

Resorbable portions of the containment device may be formed from polymer films made from synthetic materials, naturally occurring materials, modified naturally occurring materials and combinations thereof. For instance, materials suitable for synthesizing polymer films for the containment device may be formed wholly or in part from biodegradable polyurethane based on ε-caprolactone (e.g., polycaprolactone-based elastomers), which can be transformed into a film by solution casting (e.g., dip coating). The device also may be formed from a melt. Another suitable polyurethane is based on polycaprolactone-polyethylene oxide-polypropylene oxide-polyethylene oxide (Pluronic). The Pluronic may be dissolved, for example, in tetrahydrofuran.

Resorbable materials for preparing the containment device may also include polymers such as highly purified polyhydroxyacids, polyamines, polyaminoacids, copolymers of amino acids and glutamic acid, polyorthoesters, polyanhydrides, polyamides, polydioxanone, polydioxanediones, polyesteramides, polymalic acid, polyesters of diols and oxalic and/or succinic acids, polycaprolactone, copolyoxalates, polycarbonates or poly(glutamic-co-leucine). Preferably used polyhydroxyacids may comprise polycaprolactone, poly(L-lactide), poly(D-lactide), poly(L/D-lactide), poly(L/DL-lactide) polyglycolide, copolymers of lactide and glycolide of various compositions, copolymers of said lactides and/or glycolide with other polyesters, copolymers of glycolide and trimethylene carbonate, poly(glycolide-co-trimethylene carbonate), polyhydroxybutyrate, polyhydroxyvalerate, copolymers of hydroxybutyrate and hydroxyvalerate of various compositions. Other materials which may be used as additives are composite systems containing resorbable polymeric matrix and resorbable glasses and ceramics based e.g. on tricalcium phosphate and/or hydroxyapatite, admixed to the polymer before processing.

Polymer films for forming a containment device, preferably, may be specially designed to exhibit one or more desired properties. Specifically, polymer films may be formulated to have specific mechanical and chemical properties. For instance, polymer films may be designed to have a low Young's modulus; a high tensile strength; a fast resorption rate; and a high elongation at break.

Polymer films may be formulated for different degradation rates in vivo. A polymer film may be designed to substantially degrade in a matter months, weeks, or days. In an illustrative embodiment, a polyurethane film made from a polyurethane polymer may be designed to have a thickness of about 0.3 mm and may be designed to substantially degrade in vivo within one-year after implantation. In another embodiment, the polyurethane film may be designed to substantially degrade in vivo within 16 weeks after implantation. Thus, the rate of resorption and the loss of mechanical properties of the containment device in vivo may be adapted to allow maintenance of its functionality during a post-operative healing period. The rate of resorption, preferably may be controlled taking into account that such factors as polymer weight, crystallinity, polymer chain orientation, material purity, the presence of copolymer unit in the chain. The presence of voids (porosity) will affect the rate of resorption. In general the rate of resorption increases in the presence of a material with voids, pores, impurities, copolymer units. The rate of degradation decreases with the increase of polymer molecular weight, crystallinity and chain orientation.

Preferably, a suitable polymeric material may have a degradation rate in vivo in the range of 6 weeks to 24 months. Viscosity-average molecular weight of polymers to be suitable for preparation of the containment may be in the range of 30,000 to 900,000 and preferably 180,000 for elastic or semi-elastic of the containment device, and preferably 300,000 to 400,000 for harder implants.

The clinical need may also effect the formulation of the polymer and properties of the containment device. For example, a balloon or containment device which is to be implanted within a more heavily damaged bone may require a containment device that is designed to degrade more slowly in order to provide additional structural integrity to the implant or some other therapeutic benefit. A polyurethane based containment device, therefore, may include therapeutic materials which are beneficially released during the degradation of the device as part of a pre-determined and longer term therapy. For instance, the containment device may be designed to degrade substantially over a target period of several months. In an application for filling voids in bone, where it may be a primary objective to prevent the extraosseous flow of bone filler material, the biodegradable polymer may preferably have a have a low Young's modulus, a high tensile strength, fast resorption and high elongation. The rate of degradation of the film may be then be formulated to meet such a clinical need. Moreover, the polymer film may degrade in vivo to produce end products that are bio-compatible and that do not adversely affect the bone filler which has been placed inside the balloon or containment device. For example, the degradation products of a suitable urethane polymer device may include carbon dioxide, water, and diamine.

Resorbable or degradable polymeric and/or polymeric-ceramic materials for forming containment devices may have a Young's modulus in the range of 1 to 100 MPa and a tensile strength in the range of 1 to 100 MPa. The Young's modulus should preferably be in the range of 5 to 50 MPa, most preferably in the range of 15 to 25 MPa. The tensile strength should preferably be in the range of 15 to 50 MPa, most preferably in the range of 25 to 35 MPa. For example, the containment device may be formed from polyurethane materials synthesized from mixtures of polyethylene oxide with caprolactone or mixtures of polycaprolactone with triblock copolymers of ethylene oxide-propylene oxide-ethylene oxide. These materials may have an initial tensile strength in the range of 35 to 47 MPa, a moduli in the range of 22 to 31 MPa, and elongation at break in the range of 800% to 900%. Such materials may undergo rapid degradation. One embodiment of a polyurethane containment device formed from caprolactone and Pluronic (PEO-PPO-PEO) may loose about 65% of its mass after about 16 weeks of in vivo degradation.

Table 5 summarizes representative values for the physical characteristics of several foils prepared from pluronic solutions (i.e., polycaprolactone-polyethylene oxide-polypropolyne oxide). Each foil comprised an area of about 150 mm×150 mm and had a thickness of about 0.3 mm.

TABLE 5
ILLUSTRATIVE PHYSICAL PROPERTIES FOR
EXEMPLARY RESORBABLE POLYMER FOILS

| | Average | | | | |
|---|---|---|---|---|---|
| | molecular | Young's | Tensile | Elongation | Relative |
| Thickness | weight | Modulus | Strength | at Break | Resorption |
| (mm) | (Dalton) | (MPa) | (MPa) | (%) | Rate |
| 0.3 (a) | 180,000 | 22 | 34.5 | 950 | Faster |
| 0.3 (b) | 180,000 | 18 | 17 | 870 | Slower |
| 0.3 (c) | 180,000 | 10 | 46 | 660 | 1.5 year |
| 0.3 (c) | 180,000 | 13 | 48 | 770 | — |
| 0.3 (a) | 104,000 | 18 | 26 | — | — |

Notes:
(a) Pluronic (polycaprolactone-polyethylene, oxide-polypropylene, and oxide-polyethylene oxide);
(b) Polycaprolactone, and polyethylene oxide
(c) e-caprolactone.

Table 6 by contrast summarizes the preferred physical properties for a pluronic based resorbable containment device for low pressure applications. In this application, the containment device may be designed to prevent extravasation when a void in bone is filled with a filler material like bone cement.

TABLE 6
SELECTED PHYSICAL PROPERTIES FOR AN EXEMPLARY
RESORBABLE CONTAINMENT DEVICE (a)

| | | | | | Rate of |
|---|---|---|---|---|---|
| | | | | | Resorption |
| | Average | | | | (In Vivo percent |
| | molecular | Young's | Tensile | Elongation at | degradation of |
| | weight | Modulus | Strength | Break | initial mass |
| Description | (Dalton) | (Mpa) | (MPa) | (%) | after 16 weeks) |
| High | 200,000 | 30 | 50 | 1000 | 80 |
| Low | 100,000 | 5 | 15 | 600 | 50 |
| Preferred | 150,000- | 15-25 | 25-35 | 850-950 | 60-65 |
| | 190,000 | | | | |

Resorbable containment devices may be made by solution casting successive layers of polymer film onto a mandrel or mold. For example, a polyurethane polymer based on Pluronic having a viscosity-average molecular weight of 104.000 dalton may be used to prepare the polymer film. In another example, a pluronic-based polyurethane may be dissolved in tertahydofuran to prepare a 2.5 wt/vol-% solution. Other pluronic-based polymer materials having a different viscosity-average molecular weight and/or polymer concentration may also be used to prepare the polymer film for the containment device. Pluronic-based materials having a viscosity-average molecular weight of 180.000 dalton and a polymer concentration of 3%, 4%, or 5% may also be prepared. The concentration of the copolymer unit in the polymer may be in the range of about 1 to about 99% and preferably in the range of about 2.5 to about 35%. The polymeric material may have at least a partially oriented structure.

Referring to FIG. 38, the resorbable containment device 440 may be formed by solution casting a polymer film onto a mandrel or mold 450 (shown in FIG. 39). In general, the polymer concentration affects the number of layers which may need to be deposited on a mandrel or mold to build up the desired thickness of the biodegradable film. For instance, solution casting 25 to 30 polymer layers may be required to form a film having a thickness of approximately 0.3 mm on to a mandrel from a solution of pluronic material having a viscosity-average molecular weight of 104,000 dalton. The resulting polymer film may have a tensile strength of about 26 MPa and a tensile modulus of about 18 MPa. Similarly, solution casting 25 to 30 polymer layers may form a film having a thickness of approximately 0.3 mm on to a mandrel from a solution of pluronic material having, for example, a viscosity-average molecular weight of 180,000 dalton. This polymer film may have a tensile strength of about 35 MPa and a tensile modulus of about 22 MPa. Fewer than 25-30 layers of polymer film may have to be deposited on the mandrel to obtain a similar film thickness when the concentration of the polymer solution is greater.

Referring to FIG. 39, one preferred method for making a containment device from a pluronic based solution is to provide a mandrel 450 with a first portion 455 having a shape of the containment device (or balloon) to be formed and a second portion 460 attached to the first portion having a handle to allow for inserting the mold 450 into the polymer solution and rotating the mold 450 to distribute uniformly the polymer solution on the mold surface 465. Preferably, the mandrel 450 may be formed from stainless steel and may be polished to facilitate removal of the cast balloon. Alternatively, the mandrel 450 may be made from PTFE (i.e., Teflon™). Wrinkles that may form during polymer solidification on the surface of the film may result in lower tensile strength and lower tensile modulus.

Referring back to FIG. 38, the wall 470 of the device preferably may have a smooth surface and substantially uniform thickness. The resorbable containment device 440 may also have any texture, shape, or size as described above. Thus, the inflatable devices shown in FIGS. 4-16 (or molds having the similar shape) may be used for solution casting a resorbable containment device. For example, the balloon of FIG. 6 may be used as the mold for solution casting a resorbable containment device. The mold may be treated with one or more materials such as by spray or dip coating to provide a lubricating barrier for facilitating separation of the newly formed resorbable containment device from the balloon mold. For instance, materials that melt at a temperature lower than that of the polymer containment device may be used as a sacrificial layer for separating the polymer containment device from the mold.

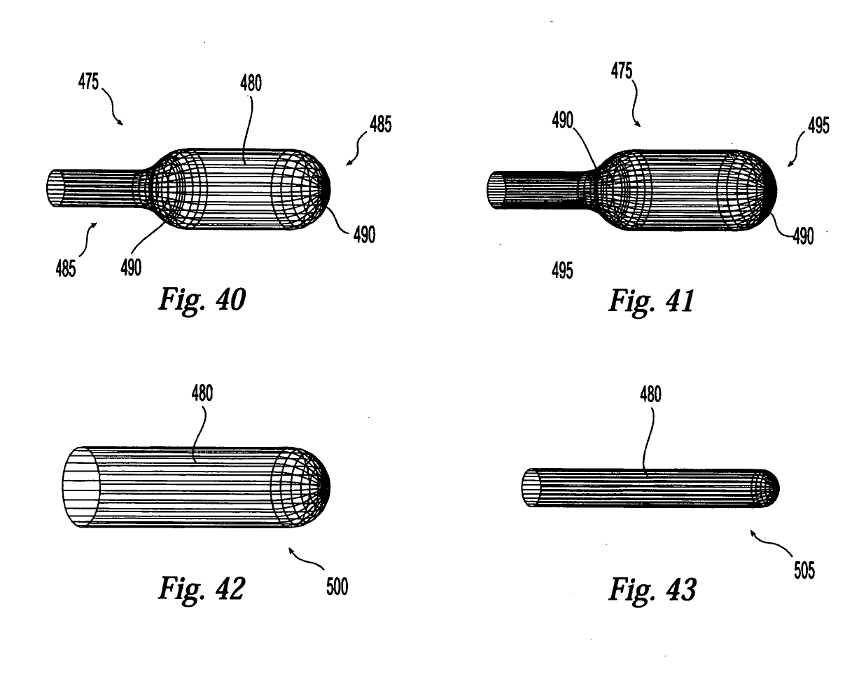

Referring to FIG. 40, a resorbable containment device 485 formed from composite materials may also be formed by solution casting. For instance, the resorbable containment device 475 may incorporate a strand 480, which may be formed from biodegradable materials, bio-inert materials, or a combination thereof. The strand 480, for example, may be formed from a medical grade metal, a polymer material, suture material, or other suitable composition. The strand 480 need not provide uniform coverage over the containment device 475 and may be placed selectively in certain areas of the containment device. One or more strands may also form a woven member. The strand 480 may cover a containment device in a uniform or non-uniform manner. For example, strand 480 with an increasingly dense pattern 485 of placement at tapering sections 490 of the containment device 475 may be used.

FIG. 41 shows an exemplary containment device 475 having strand 480 with a pattern 495 of placement having a greater density of strand 480 than the device shown in FIG. 40.

FIGS. 42 and 43 show exemplary embodiments of preformed socks 500, 505 of strand 480 which may be placed on a mold like the mold 450 shown in FIG. 39. The strand 480 may otherwise be wrapped onto the mold 450 from a spool of material. The spool may contain a single thread, or filament, or may comprise more than one thread which have been woven together to form a composite chord. Similarly, the spool may contain coated or coaxial threads and chords.

A resorbable containment device 440 may then be solution cast on the mold 450 and strand 480. The strand 480 may also be placed over a partially or completely cast containment device. Additional solution casting of resorbable polymer may then be applied to partially or completely cover the strand. Thus, a resorbable containment device 440 may be formed with a strand 480 located within the wall 445 of the resorbable containment device, on the inside surface of the containment device, or on the outside surface 470 of the containment device.

Resorbable containment devices may also be made from individually cast molds. Individually formed sections of resorbable polymer may be applied directly to bone or may be combined to create barriers or larger containment structures. For instance, individual pieces of a composite containment device may be joined and/or sealed together for low pressure filling with bone cement in vivo, by drip coating the joints between the individual pieces of the composite device with pluronic based solution. Alternatively, any suitable adhesive may be used to join pieces into a unitary structure. Individual sections of resorbable polymer may also be stitched together with suture material. The stitching may be designed to provide a leak free structure or may require additional sealing of the seams. Automated spray coating of molds formed by CAD/CAM and other known processes may be used to form containment devices of a wide variety of shapes, sizes and materials. Resorbable containment devices may also be produced using standard techniques of polymer processing, mainly by injection-molding, compression-molding and in-mold polymerization.

While the above invention has been described with reference to certain preferred embodiments, it should be kept in mind that the scope of the present invention is not limited to these embodiments. For example, the containment device may be formed with a strand that extends from the resorbable polymer to form a free end. The free end may be used to secure the containment device or tie off the opening. The embodiments above can also be modified so that some features of one embodiment are used with the features of another embodiment. One skilled in the art may find variations of these preferred embodiments which, nevertheless, fall within the spirit of the present invention, whose scope is defined by the claims set forth below.

1. A device for containing material inside bone comprising:
a barrier member configured and adapted for insertion into bone, the barrier member having inner and outer surfaces, the inner surface defining a space,
wherein the barrier member is capable of preventing fluid within the space from passing through the inner surface to the outer surface, and the barrier member comprises a polyurethane polymer based on at least one of the group consisting of capralactone and Pluronic, such that the barrier member is capable of degrading into biologically compatible substances in vivo.

2. The device of claim 1, wherein the flexible material comprises a plasticizer.

3. The device of claim 2, wherein the flexible material is a resorbable material.

4. The device of claim 3, wherein the flexible material has high elongation.

5. The device of claim 1, wherein the flexible material has an elongation at break of between about 1,000-percent and about 2,000-percent.

6. The device of claim 1, wherein the flexible material has a Young's modulus between about 10 MPa and about 100 MPa.

7. The device of claim 1, wherein the flexible material has a tensile strength at break between about 5 to about 100 MPa.

8. The device of claim 1, wherein the barrier member comprises a plurality of layers.

9. The device of claim 8, wherein the barrier member has a tensile strength between about 15 and about 50 MPa.

10. The device of claim 9, wherein the barrier member has a tensile strength between about 25 MPa and about 35 MPa.

11. The device of claim 8, wherein the barrier member has a Young's modulus of between about 5 MPa and about 30 MPa.

12. The device of claim 11, wherein the barrier member has a Young's modulus of between about 15 MPa and about 25 MPa.

13. The device of claim 11, wherein the barrier member has an elongation at break of between about 600-percent and 1000-percent.

14. The device of claim 13, wherein the barrier member has an elongation at break of between about 850-percent and 950-percent.

15. The device of claim 13, wherein the barrier member has an average molecular weight of between about 100,000 and about 200,000 dalton.

16. The device of claim 15, wherein the barrier member has an average molecular weight of between about 150,000 and about 190,000 dalton.

17. The device of claim 1, wherein the barrier member has mass and the mass degrades in vivo after the device is implanted into bone.

18. The device of claim 17, wherein more than about 60-percent of the mass degrades after about 2 weeks of in vivo degradation.

19. The device of claim 18, wherein the barrier member has a thickness of about 0.5 mm.

20. The device of claim 19, wherein the mass degrades in vivo to produce carbon dioxide, water and diamine.

21. A method of forming a resorbable containment device comprising:
providing a mold;
providing a polymer;
depositing a plurality of layers of the polymer on the mold to form the resorbable containment device; and
removing the resorbable containment device from the mold.

22. The method of claim 21 further comprising treating the mold with at least one lubricating material to provide a lubricating barrier for facilitating removal of the resorbable containment device from the mold.

23. The method of claim 22 further comprising spraying the mold with the at least one lubricating material.

24. The method of claim 22 further comprising dip coating the mold in at least one lubricating material.

25. The method of claim 21 further comprising positioning at least one strand on the mold.

26. The method of claim 25 further comprising forming a woven member with the at least one strand.

27. The method of claim 21 further comprising positioning at least one strand on one of a partially and completely formed resorbable containment device.

28. The method of claim 21 further comprising:
positioning at least one strand on a partially formed resorbable containment device; and
depositing additional polymer over the at least one strand.

29. The method of claim 21 further comprising forming the mold of stainless steel and polishing the stainless steel.

30. The method of claim 21 further comprising forming the mold of PTFE.

31. The method of claim 21, wherein the step of depositing a plurality of layers of the polymer on the mold comprises solution casting.
